(19) United States
(12) Patent Application Publication
(10) Pub. No.: US 2012/0052015 A1
(43) Pub. Date: Mar. 1, 2012

(54) END-CAPPED POLYMERS

(75) Inventors: Lei Yu, Carlsbad, CA (US); Hai Wang, San Diego, CA (US); Kwok Yin Tsang, Irvine, CA (US); Yi Jin, Carlsbad, CA (US); Sang Van, San Diego, CA (US)

(73) Assignee: Nitto Denko Corporation, Osaka (JP)

(21) Appl. No.: 13/213,037

(22) Filed: Aug. 18, 2011

Related U.S. Application Data
(60) Provisional application No. 61/377,421, filed on Aug. 26, 2010.

(51) Int. Cl. A61K 47/48; A61K 49/12; A61P 35/00; C08G 69/48

(52) U.S. Cl. 424/9.3; 525/432; 525/426; 525/420; 424/78.17

(57) ABSTRACT

Polymers that can include an end-cap selected from Formula (I) and/or Formula (II) and a recurring unit of the Formulae (III), and/or (IV). The polymers can include various substituents such as drug, targeting agents, stabilizing agents, imaging agent and polydentate ligands. Such polymer can be useful for variety of drug, targeting, stabilizing and/or imaging agent delivery applications.

### CROSS-REFERENCE TO RELATED APPLICATIONS

This application claims priority to U.S. Provisional Patent Application No. 61/377,421, entitled “END-CAPPED POLYMERS” filed Aug. 26, 2010, which is incorporated herein by reference in its entirety, including any drawings.

### BACKGROUND

1. Field

This application relates generally to end-capped polymers and methods of making the same. The polymers described herein are useful for a variety of drug, biomolecule, and imaging agent delivery applications. Also disclosed are methods of polymers to treat, diagnose, and/or image a subject.

2. Description

A variety of systems have been used for the delivery of drugs, biomolecules, and imaging agents. For example, such systems include capsules, liposomes, microparticles, nanoparticles, and polymers.

A variety of polyester-based biodegradable systems have been characterized and studied. Polylactic acid (PLA), polyglycolic acid and their copolymers polylactic-co-glycolic acid (PLGA) are some examples of well-characterized biomaterials with regard to design and performance for drug-delivery applications. See Uhrich, K. E.; Cannizzaro, S. M.; Langer, R. S. and Shakeshelf, K. M. “Polymeric Systems for Controlled Drug Release,” Chem. Rev. 1999, 99, 3181-3198 and Panyam J, Labhasetwar V. “Biodegradable nanoparticles for drug and gene delivery to cells and tissue,” Adv. Drug. Deliv. Rev. 2003, 55, 329-47. Also, 2-hydroxypropyl methacrylate (HPMA) has been widely used to create a polymer for drug-delivery applications. Biodegradable systems based on polyorthoesters have also been investigated. See Heller, J.; Barr, J.; Ng, S. Y.; Abdellauoi, K. S. and Gurny, R. “Poly(ortho esters): synthesis, characterization, properties and uses.” Adv. Drug Del. Rev. 2002, 54, 1015-1039. Polyanhydride systems have also been investigated. Such polyanhydrides are typically biocompatible and may degrade in vivo into relatively non-toxic compounds that are eliminated from the body as metabolites. See Kumar, N.; Langer, R. S, and Domb, A. J. “Polyanhydrides: an overview,” Adv. Drug Del. Rev. 2002, 54, 889-91.

Amino acid-based polymers have also been considered as a potential source of new biomaterials. Poly-amino acids having good biocompatibility have been investigated to deliver low molecular-weight compounds. A relatively small number of polyglutamic acids and copolymers have been identified as candidate materials for drug delivery. See Bourke, S. L. and Kohn, J. “Polymers derived from the amino acid L-tyrosine: polycarbonates, polyarylates and copolymers with poly(ethylene glycol).” Adv. Drug Del. Rev., 2003, 55, 447-466.

Administered hydrophobic anticancer drugs, therapeutic proteins, and polypeptides often suffer from poor bio-availability. Such poor bio-availability may be due to incompatibility of bi-phasic solutions of hydrophobic drugs and aqueous solutions and/or rapid removal of these molecules from blood circulation by enzymatic degradation. One technique for increasing the efficacy of administered proteins and other small molecule agents entails conjugating the administered agent with a polymer, such as a polyethylene glycol (“PEG”) molecule, that can provide protection from enzymatic degradation in vivo. Such “PEGylation” often improves the circulation time and, hence, bio-availability of an administered agent.

PEG has shortcomings in certain respects, however. For example, because PEG is a linear polymer, the steric protection afforded by PEG is limited, as compared to branched polymers. Another shortcoming of PEG is that it is generally amenable to derivatization at its two terminals. This limits the number of other functional molecules (e.g. those helpful for protein or drug delivery to specific tissues) that can be conjugated to PEG.

Polyglutamic acid (PGA) is another polymer of choice for solubilizing hydrophobic anticancer drugs. Many anti-cancer drugs conjugated to PGA have been reported. See Chun Li. “Poly(L-glutamic acid)-anticancer drug conjugates.” Adv. Drug Del. Rev., 2002, 54, 695-713. However, none are currently FDA-approved.

Paclitaxel, extracted from the bark of the Pacific Yew tree, is a FDA-approved drug for the treatment of ovarian cancer and breast cancer. Wani et al. “Plant antitumor agents. VI. The isolation and structure of taxol, a novel antileukemic and antitumor agent from Taxus brevifolia,” J. Am. Chem. Soc. 1971, 93, 2325-7. However, like other anti-cancer drugs, pacilitaxel suffers from poor bio-availability due to its hydrophobicity and insolubility in aqueous solution. One way to solubilize pacilitaxel is to formulate it in a mixture of Cremophor-EL and dehydrated ethanol (1:1, v/v). Sparreboom et al. “Cremophor EL-mediated Alteration of Paclitaxel Distribution in Human Blood: Clinical Pharmacokinetic Implications,” Cancer Research, 1999, 59, 1454-1457. This formulation is currently commercialized as Taxol® (Bristol-Myers Squibb). Another method of solubilizing paclitaxel is by emulsification using high-shear homogenization. Constantinides et al. “Formulation Development and Antitumor Activity of a Filter-Sterilizable Emulsion of Paclitaxel,” Pharmaceutical Research 2000, 17, 175-182. Recently, polymer-paclitaxel conjugates have been advanced in several clinical trials. Ruth Duncan “The Dawning era of polymer therapeutics,” Nature Reviews Drug Discovery 2003, 2, 347-360. More recently, paclitaxel has been formulated into nano-particles with human albumin protein and has been used in clinical studies. Damascelli et al. “Intraarterial chemotherapy with polyoxyethylated castor oil free paclitaxel, incorporated in albumin nanoparticles (ABI-007): Phase II study of patients with squamous cell carcinoma of the head and neck and anal canal: preliminary evidence of clinical activity.” Cancer, 2001, 92, 2592-602, and Ibrahim et al. “Phase I and pharmacokinetic study of ABI-007, a Cremophor-free, protein-stabilized, nanoparticle formulation of paclitaxel,” Clin. Cancer Res. 2002, 8, 1038-44. This formulation is currently commercialized as Abraxane (American Pharmaceutical Partners, Inc.).

Magnetic resonance imaging (MRI) is an important tool in diagnosis and staging of disease because it is non-invasive and non-irradiating. See Bulte et al. “Magnetic resonance microscopy and histology of the CNS,” Trends in Biotechnology, 2002, 20, S24-S28). Although images of tissues can be obtained, MRI with contrast agents significantly improves its resolution. However, paramagnetic metal ions suitable for MRI contrast agents are often toxic. One of the methods to reduce toxicity is to chelate these metal ions with polydentate molecules such as diethylenetriamine pentaacetate molecules (DTPA). Gd-DTPA was approved by FDA in 1988 for clinical uses, and it is currently commercialized as Magnevist®. Other Gd-chelates were approved by FDA and commercialized, and many others are under development. See Caravan et al. “Gadolinium(III) Chelates as MRI Contrast Agents: Structure, Dynamics, and Applications,” Chem. Rev. 1999, 99, 2293-2352.

However, Gd-DTPA is not ideal for targeting tumor tissues because it lacks specificity. When Gd-DTPA is administered via IV injection, it spontaneously and rapidly diffuses into extravascular space of the tissues. Thus, large amounts of contrast agents are usually required to produce reasonable contrast images. In addition, it is quickly eliminated via kidney filtration. To avoid the diffusion and the filtration, macromolecular MRI contrast agents have been developed. See Caravan et al. “Gadolinium(III) Chelates as MRI Contrast Agents Structure, Dynamics, and Applications,” Chem. Rev. 1999, 99, 2293-2352. These macromolecular-MRI contrast agents include protein-MRI chelates, polysaccharide-MRI chelates, and polymer-MRI chelates. See Lauffer et al. “Preparation and Water Relaxation Properties of Proteins Labeled with Paramagnetic Metal Chelates,” Magn. Reson. Imaging 1985, 3, 11-16; Sirlin et al. “Gadolinium-DTPA-Dextran: A Macromolecular MR Blood Pool Contrast Agent,” Acad. Radiol. 2004, 11, 1361-1369; Lu et al. “Poly(L-glutamic acid) Gd(III)-DOTA Conjugate with a Degradable Spacer for Magnetic Resonance Imaging,” Bioconjugate Chem. 2003, 14, 715-719; and Wen et al. “Synthesis and Characterization of Poly(L-glutamic acid) Gadolinium Chelate: A New Biodegradable MRI Contrast Agent,” Bioconjugate Chem. 2004, 15, 1408-1415.

Recently, tissue-specific MRI contrast agents have been developed. See Weinmann et al. “Tissue-specific MR contrast agents.” Eur. J. Radiol. 2003, 46, 33-44. However, tumor-specific MRI contrast agents have not been reported in clinical applications. Nano-size particles have been reported to target tumor-tissues via an enhanced permeation and retention (EPR) effect. See Brannon-Peppas et al. “Nanoparticle and targeted systems for cancer therapy.” ADDR, 2004, 56, 1649-1659).

### SUMMARY

Some embodiments described herein relate to a polymer that can include an end-cap selected from Formula (I) and Formula (II) and at least one recurring unit selected from Formula (III) and Formula (IV) as set forth herein, wherein: m can be independently 1 or 2; R¹ and R⁵ can be independently selected from an optionally substituted C₁₋₁₀ alkyl and an optionally substituted C₆₋₂₀ aryl; A¹, A² and A³ can be independently oxygen or NR⁹, wherein R⁹ can be hydrogen or C₁₋₄ alkyl; R², R³ and R⁴ can be independently selected from hydrogen, C₁₋₁₀ alkyl, C₆₋₂₀ aryl, ammonium, alkali metal, a polydentate ligand, a polydentate ligand precursor with protected oxygen atoms and a first compound that comprises an agent; n can be independently 1 or 2; p can be 0 or an integer ≧1; s can be 0 or an integer ≧1; provided that the sum of p+s is ≧1; each A⁴, each A⁵ and each A⁶ can be independently oxygen or NR¹⁰, wherein each R¹⁰ can be hydrogen or C₁₋₄ alkyl; and each R⁶, each R⁷ and each R⁸ can be independently selected from hydrogen, C₁₋₁₀ alkyl, C₆₋₂₀ aryl, ammonium, alkali metal, a polydentate ligand, a polydentate ligand precursor with protected oxygen atoms and a second compound that comprises an agent; and each agent can be independently selected from a drug, a targeting agent, an optical imaging agent, a magnetic resonance imaging agent, and a stabilizing agent.

Other embodiments described herein relates to a method of making a polymer as described herein that can include dissolving or partially dissolving a polymeric reactant that can include at least one recurring unit selected from Formula (V) and Formula (VI), as set forth herein, in a solvent to form a dissolved or partially dissolved polymeric reactant; wherein: o can be independently 1 or 2; A⁷, A⁸, and A⁹ can be oxygen; and R¹⁵, R¹⁶ and R¹⁷ can be independently selected from hydrogen, ammonium, and an alkali metal; and reacting the dissolved or partially dissolved polymeric reactant with a second reactant, wherein the second reactant can include a compound of Formula (VII), as set forth herein, wherein R¹⁸ and R¹⁹ can be independently selected from an optionally substituted C₁₋₁₀ alkyl and an optionally substituted C₆₋₂₀ aryl.

Other embodiments described herein relate to a pharmaceutical composition that can include a polymer described herein, and at least one selected from a pharmaceutically acceptable excipient, a carrier and a diluent.

Still other embodiments described herein relate to a method of treating or ameliorating a disease or condition that can include administering an effective amount of a polymer or a pharmaceutical composition described herein to a subject in need thereof.

Some embodiments described herein relate to the use of a polymer or a pharmaceutical composition described herein in the preparation of a medicament for treating or ameliorating a disease or condition.

Other embodiments described herein relate to a method of diagnosing a disease or condition that can include administering an effective amount of a polymer or a pharmaceutical composition described herein to a subject in need thereof.

Still other embodiments described herein relate to the use of a polymer or a pharmaceutical composition described herein in the preparation of a medicament for diagnosing a disease or condition.

Yet still embodiments described herein relate to a method of imaging a portion of tissue that can include contacting the portion of tissue with an effective amount of a polymer or a pharmaceutical composition described herein.

Some embodiments described herein relate to the use of a polymer or a pharmaceutical composition described herein in the preparation of a medicament for imaging a portion of tissue.

These and other embodiments are described in greater detail below.

### BRIEF DESCRIPTION OF THE DRAWINGS

FIG. 1 illustrates a general scheme for preparing non-capped PGA-PTX and capped PGA-PTX.

FIG. 2 provides pictorial abbreviations for paclitaxel and poly(L-glutamic acid) (NH₂—PGA-COOH).

FIG. 3 illustrates a general reaction scheme for the preparation of a PGA-paclitaxel conjugate and a PGA-paclitaxel aggregate.

FIG. 4 illustrates a general reaction scheme for the preparation of a capped PGA-COOH and capped PGA-paclitaxel conjugate (cPGA-PTX).

FIG. 5 illustrates chromatograms and summary tables from the molecular weight experiments of non-capped sPGA-PTX 3, capped sPGA-PTX 4, and Capped PGA-PTX 5.

FIG. 6 illustrates a general reaction scheme for the preparation of poly-L-(glutamyl-glutamine) acid (PGGA) and poly-L-(glutamyl-glutamine) acid aggregate (PGGA aggregate).

FIG. 7 illustrates a general reaction scheme for the preparation of capped PGA-COOH and capped poly-L-(glutamyl-glutamine) acid (cPGGA).

FIG. 8 illustrates chromatograms and summary tables from the molecular weight experiments of non-capped sPGGA 6.

FIG. 9 illustrates chromatograms and summary tables from the molecular weight experiments of capped PGGA 7.

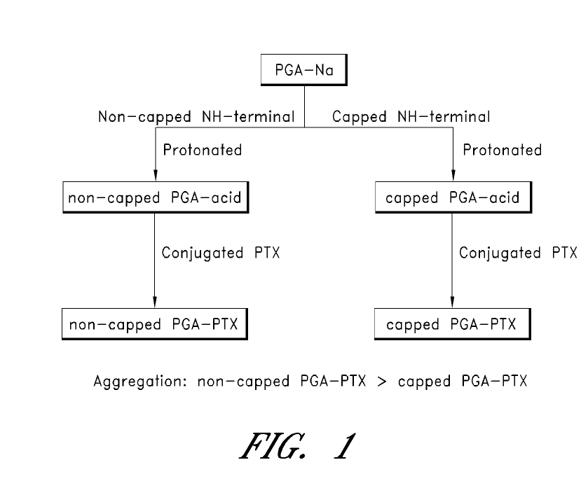

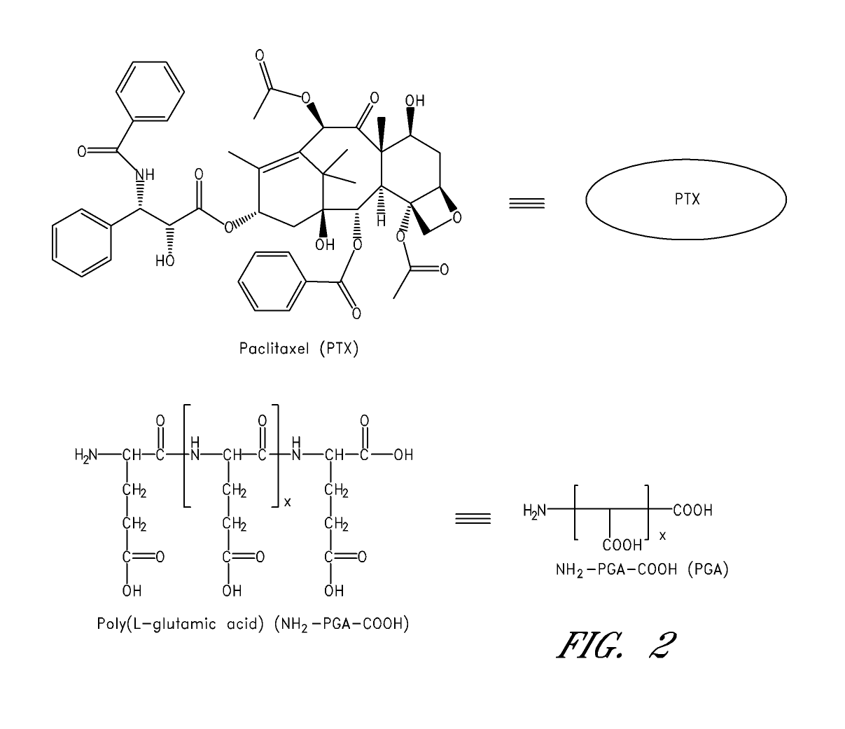

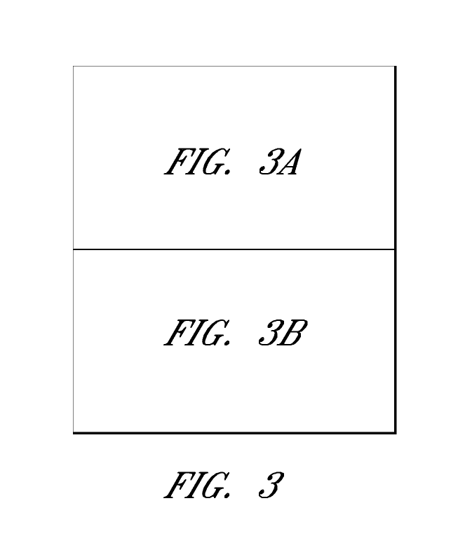

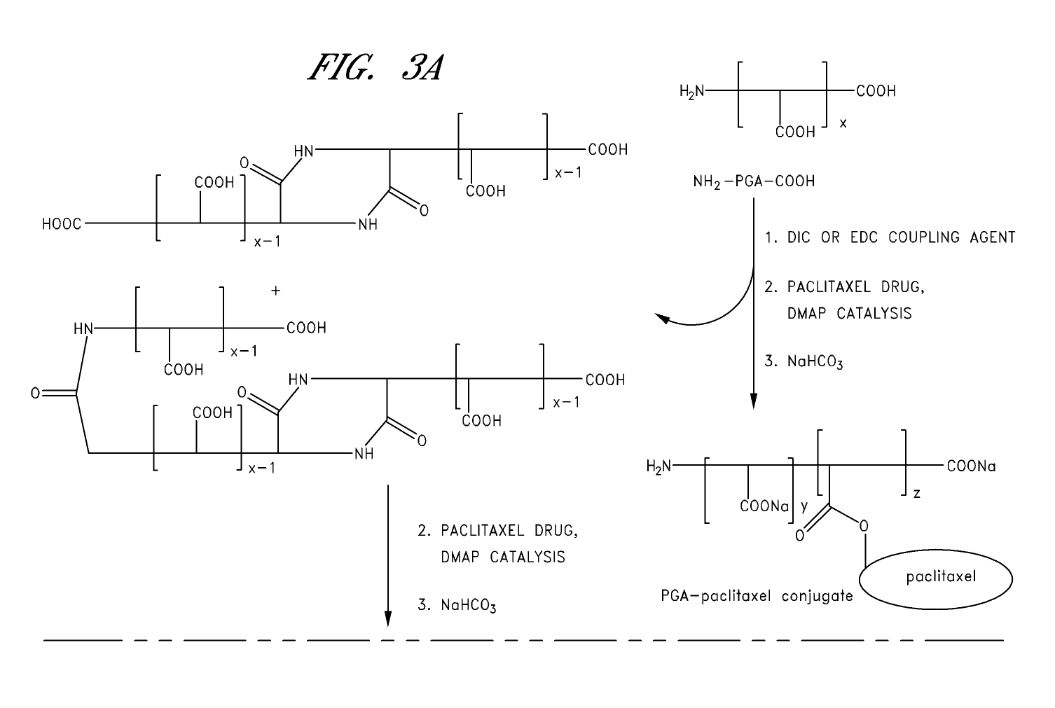

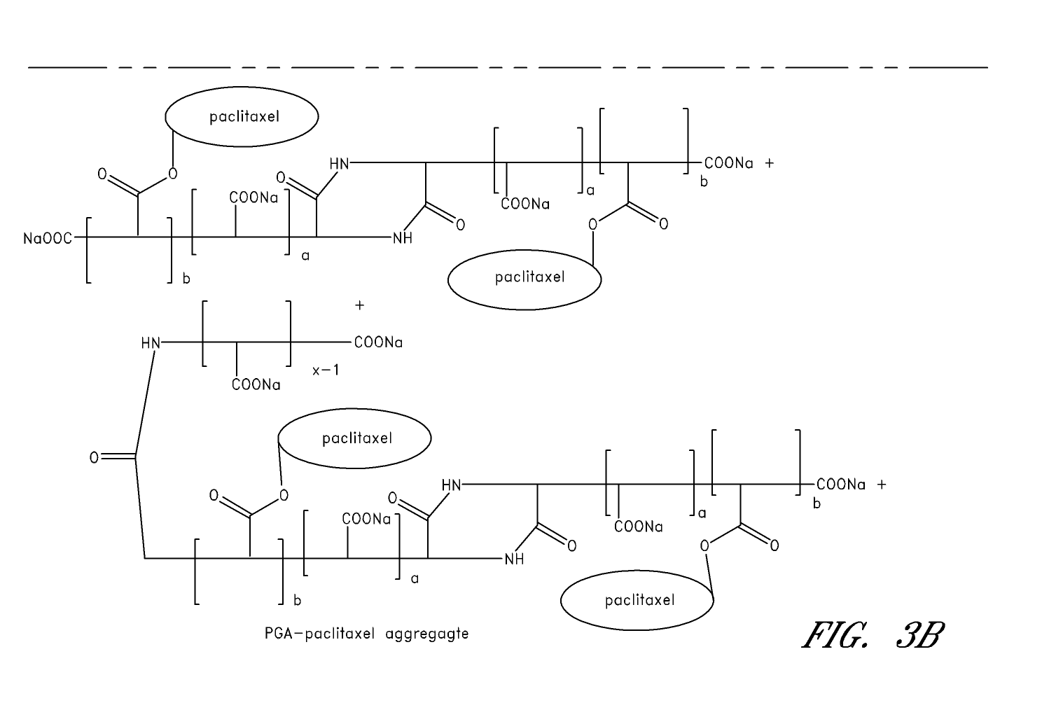

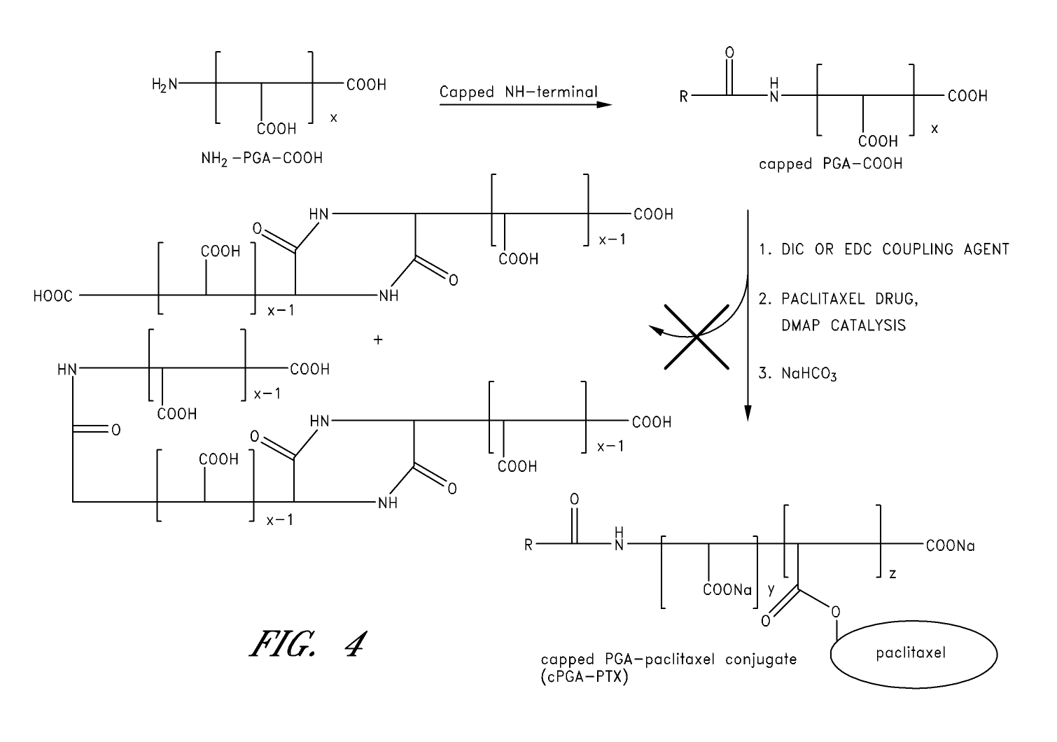

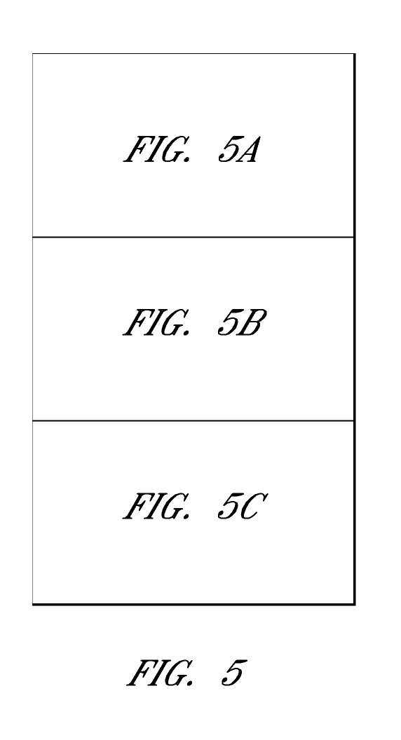

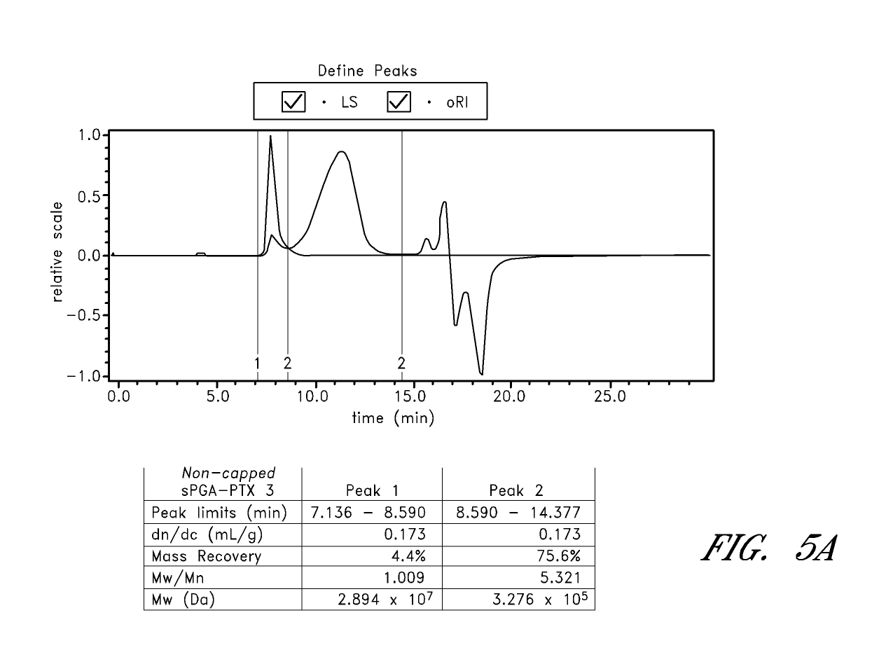

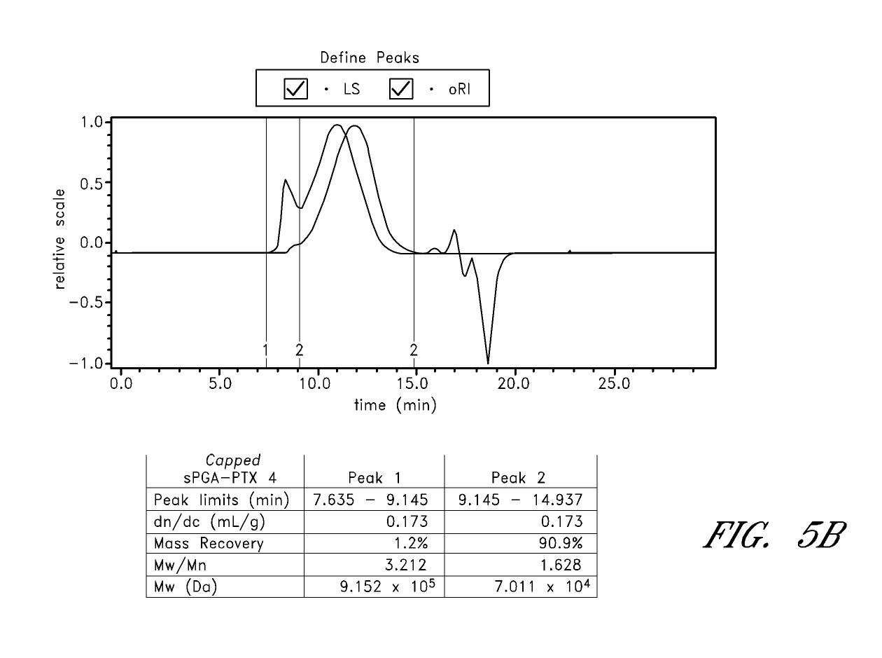

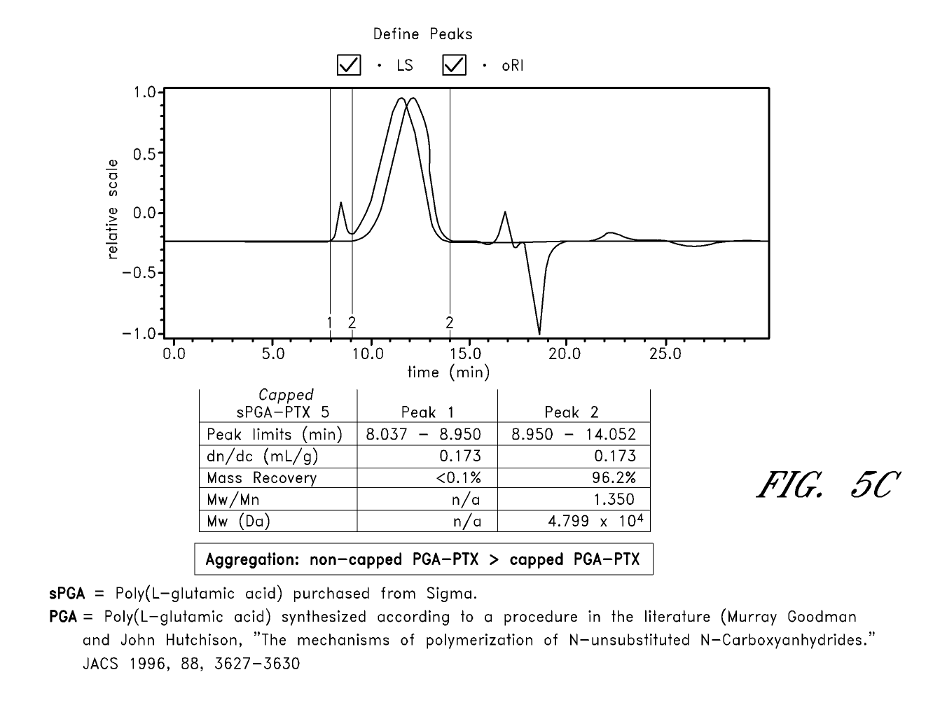

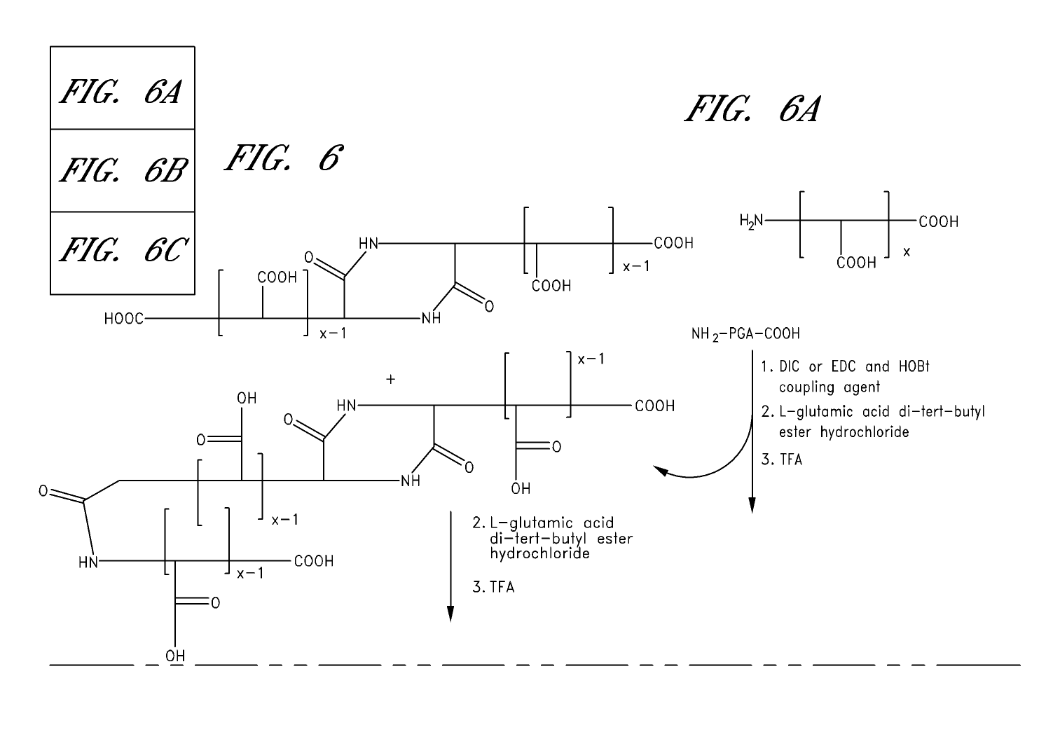

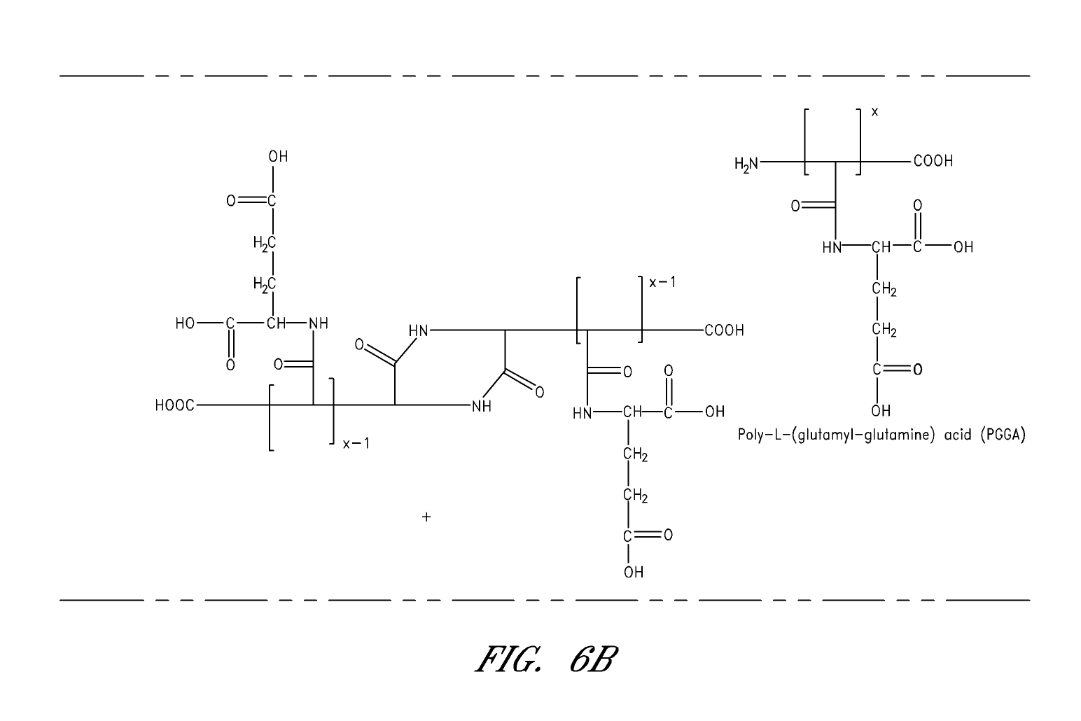

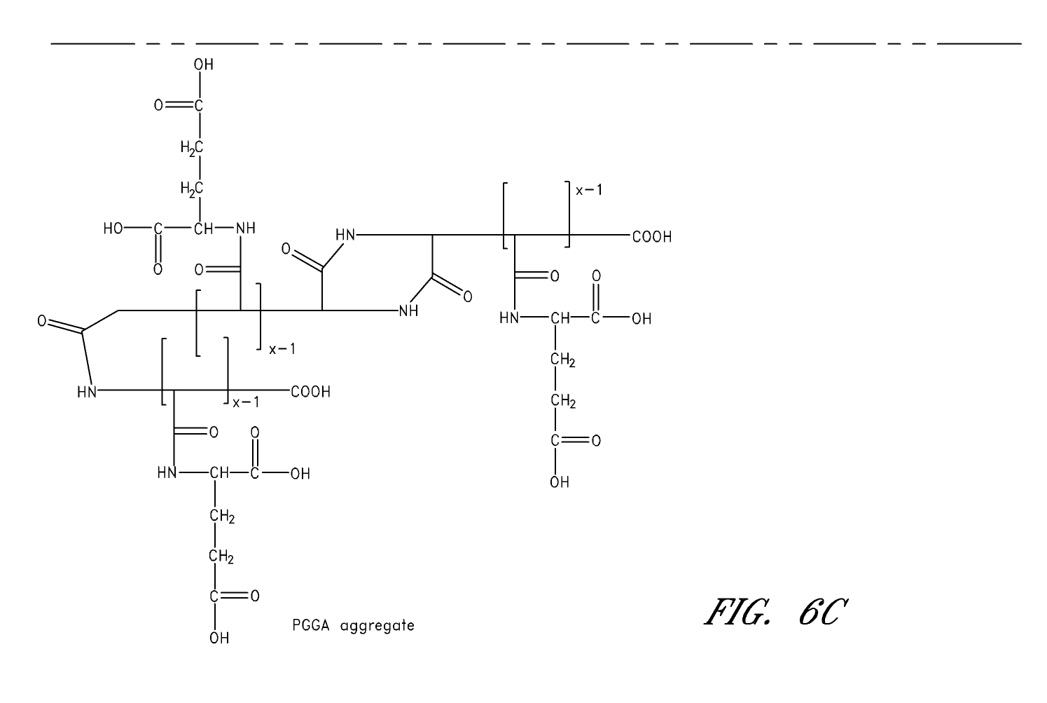

### DETAILED DESCRIPTION

A variety of polymer-based systems have been developed for the delivery of drugs, biomolecules, and imaging agents. One of the problems with some of the polymers used in polymer-delivery systems is the polymer can aggregate together during its synthesis. The aggregation can result in the polymer having a higher than desired average molecular weight. In some embodiments, by end-capping the polymer, the average molecular weight of the polymer can be more easily controlled. Furthermore, aggregation that takes place during the synthesis of the polymer can be minimized. In some embodiments, by end-capping the polymer, the polymer can be less polydisperse.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

The alkyl group may be substituted or unsubstituted. When substituted, the substituent group(s) is(are) one or more group(s) individually and independently selected from C₂₋₆ alkenyl, C₂₋₆ alkynyl, C₃₋₇ cycloalkyl, C₃₋₇ cycloalkenyl, C₃₋₇ cycloalkynyl, C₆₋₁₀ aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl (e.g., mono-, di- and tri-haloalkyl), haloalkoxy (e.g., mono-, di- and tri-haloalkoxy), trihalomethanesulfonyl, trihalomethanesulfonamido, and amino, including mono- and di-substituted amino groups, and the protected derivatives thereof.

As used herein, “aryl” refers to a carbocyclic (all carbon) monocyclic, bicyclic and tricyclic aromatic ring systems that has a fully delocalized pi-electron system throughout all of the rings. Examples of aryl groups include, but are not limited to, benzene, naphthalene and azulene. An aryl group may be substituted or unsubstituted. When substituted, hydrogen atom(s) are replaced by substituent group(s) that is(are) one or more group(s) independently selected from C₂₋₆ alkenyl, C₂₋₆ alkynyl, C₃₋₇ cycloalkyl, C₃₋₇ cycloalkenyl, C₃₋₇ cycloalkynyl, C₆₋₁₀ aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl (e.g., mono-, di- and tri-haloalkyl), haloalkoxy (e.g., mono-, di- and tri-haloalkoxy), trihalomethanesulfonyl, trihalomethanesulfonamido, and amino, including mono- and di-substituted amino groups, and the protected derivatives thereof, unless the substituent groups are otherwise indicated.

The term “ester” is used herein in its ordinary sense, and thus includes a chemical moiety with formula —(R)ₙ—COOR′, where R and R′ are independently selected from C₁₋₆ alkyl, C₃₋₇ cycloalkyl, C₆₋₁₀ aryl, heteroaryl (bonded through a ring carbon) and heteroalicyclic (bonded through a ring carbon), and where n is 0 or 1.

The term “amide” is used herein in its ordinary sense, and thus includes a chemical moiety with formula —(R)ₙ—C(O)NHR′ or —(R)ₙ—NHC(O)R′, where R and R′ are independently selected from C₁₋₆ alkyl, C₃₋₇ cycloalkyl, C₆₋₁₀ aryl, heteroaryl (bonded through a ring carbon) and heteroalicyclic (bonded through a ring carbon), and where n is 0 or 1. An amide may be included in an amino acid or a peptide molecule attached to drug molecule as described herein, thereby forming a prodrug.

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents. If no substituents are indicated, it is meant that the indicated “optionally substituted” or “substituted” group may be substituted with one or more group(s) individually and independently selected from C₁₋₆ alkyl, C₂₋₆ alkenyl, C₂₋₆ alkynyl, C₃₋₇ cycloalkyl, C₃₋₇ cycloalkenyl, C₃₋₇ cycloalkynyl, C₆₋₁₀ aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl (e.g., mono-, di- and tri-haloalkyl), haloalkoxy (e.g., mono-, di- and tri-haloalkoxy), trihalomethanesulfonyl, trihalomethanesulfonamido, and amino, including mono- and di-substituted amino groups, and the protected derivatives thereof.

Any amine, hydroxy, or carboxyl side chain on the compounds disclosed herein can be esterified or amidified. The procedures and specific groups to be used to achieve this end are known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

A “paramagnetic metal chelate” is a complex wherein a ligand is bound to a paramagnetic metal ion. Examples include, but are not limited to, 1,4,7,10-Tetraazacyclododecane-1,4,7,10-tetraacetic acid (DOTA)-Gd(III), DOTA-Yttrium-88, DOTA-Indium-111, diethylenetriaminepentaacetic acid (DTPA)-Gd(III), DTPA-yttrium-88, DTPA-Indium-111.

A “polydentate ligand” is a ligand that can bind itself through two or more points of attachment to a metal ion through, for example, coordinate covalent bonds. Examples of polydentate ligands include, but are not limited to, diethylenetriaminepentaacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE),2,4-pentanedione (acac), and ethanedioate (ox).

A “polydentate ligand precursor with protected oxygen atoms” is a polydentate ligand comprising oxygen atoms, such as the single-bonded oxygen atoms of carboxyl groups, that are protected with suitable protecting groups. Suitable protecting groups include, but are not limited to, lower alkyls, benzyls, and silyl groups.

A “stabilizing agent” is a substituent that enhances bioavailability and/or prolongs the half-life of a carrier-drug conjugate in vivo by rendering it more resistant to hydrolytic enzymes and less immunogenic. An example of a suitable stabilizing agent is polyethylene glycol (PEG).

An “amino acid with protected oxygen atoms” is an amino acid wherein one or more of the single-bonded oxygen atom(s) of the carboxylic acid group(s) are protected with suitable protecting groups. Suitable protecting groups include, but are not limited to, lower alkyls, benzyls, and silyl groups.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound described herein having one or more double bond(s) generating geometrical isomers that can be defined as E or Z, each double bond may independently be E or Z a mixture thereof. Likewise, all tautomeric forms are also intended to be included.

Some embodiments described herein relate to a polymer that can include an end-cap selected from Formula (I) and Formula (II) and at least one recurring unit selected from Formula (III) and Formula (IV):

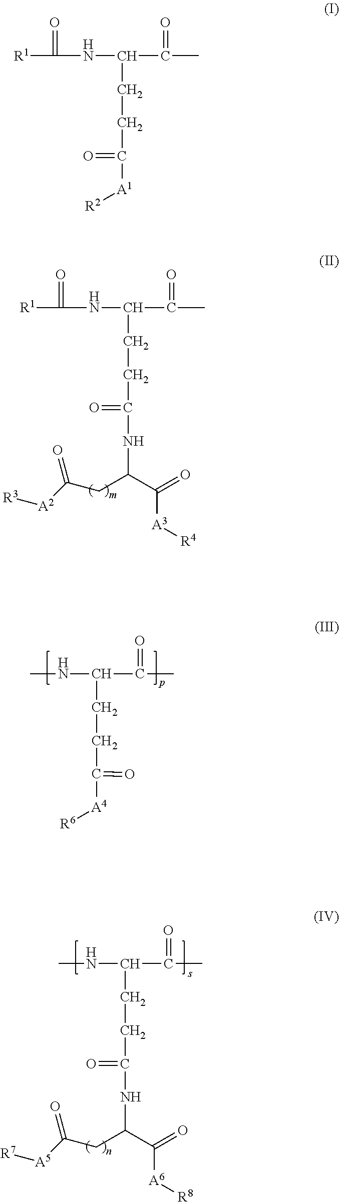

wherein m can be independently 1 or 2; R¹ and R⁵ can be independently selected from an optionally substituted C₁₋₁₀ alkyl and an optionally substituted C₆₋₂₀ aryl; A¹, A², and A³ can be independently oxygen or NR⁹, wherein R⁹ can be hydrogen or C₁₋₄ alkyl; R², R³ and R⁴ can be independently selected from hydrogen, C₁₋₁₀ alkyl, C₆₋₂₀ aryl, ammonium, alkali metal, a polydentate ligand, a polydentate ligand precursor with protected oxygen atoms and a first compound that comprises an agent; n can be independently 1 or 2; p can be 0 or an integer ≧1; s can be 0 or an integer ≧1; provided that the sum of p+s is ≧1; each A⁴, each A⁵ and each A⁶ can be independently oxygen or NR¹⁰, wherein each R¹⁰ can be hydrogen or C₁₋₄ alkyl; and each R⁶, each R⁷ and each R⁸ can be independently selected from hydrogen, C₁₋₁₀ alkyl, C₆₋₂₀ aryl, ammonium, alkali metal, a polydentate ligand, a polydentate ligand precursor with protected oxygen atoms and a second compound that comprises an agent; and each agent can be independently selected from a drug, a targeting agent, an optical imaging agent, a magnetic resonance imaging agent, and a stabilizing agent.

In some embodiments, the end-cap can be Formula (I), p can be ≧1, and s can be 0. In other embodiments, the end-cap can be Formula (I), s can be ≧1, and p can be 0. In still other embodiments, the end-cap can be Formula (I), p can be ≧1, and s can be ≧1. In yet still other embodiments, the end-cap can be Formula (II), p can be ≧1, and s can be 0. In some embodiments, the end-cap can be Formula (II), s can be ≧1, and p can be 0. In other embodiments, the end-cap can be Formula (II), p can be ≧1, and s can be ≧1.

The number of recurring units present in the polymer (for example, a polymer that can include recurring units of Formula (III) and/or (IV)) can vary over a wide range. In some embodiments, the polymer can include ≧99% mole percent of the recurring unit of Formula (III) based on ratio of total moles of recurring units of Formula (III) to the total moles of recurring units in the polymer. In other embodiments, the polymer can include ≧80% mole percent of the recurring unit of Formula (III) based on ratio of total moles of recurring units of Formula (III) to the total moles of recurring units in the polymer. In still other embodiments, the polymer can include ≧50% mole percent of the recurring unit of Formula (III) based on ratio of total moles of recurring units of Formula (III) to the total moles of recurring units in the polymer. In yet still other embodiments, the polymer can include ≧30% mole percent of the recurring unit of Formula (III) based on ratio of total moles of recurring units of Formula (III) to the total moles of recurring units in the polymer.

In some embodiments, the polymer can include ≧99% mole percent of the recurring unit of Formula (IV) based on ratio of total moles of recurring units of Formula (IV) to the total moles of recurring units in the polymer. In other embodiments, the polymer can include ≧80% mole percent of the recurring unit of Formula (IV) based on ratio of total moles of recurring units of Formula (IV) to the total moles of recurring units in the polymer. In still other embodiments, the polymer can include ≧50% mole percent of the recurring unit of Formula (IV) based on ratio of total moles of recurring units of Formula (IV) to the total moles of recurring units in the polymer. In yet still other embodiments, the polymer can include ≧30% mole percent of the recurring unit of Formula (IV) based on ratio of total moles of recurring units of Formula (IV) to the total moles of recurring units in the polymer.

In some embodiments, the polymer can include ≧99% mole percent of the recurring units Formula (III) and Formula (IV) based on ratio of total moles of recurring units of Formula (III) and recurring units of Formula (IV) to the total moles of recurring units in the polymer. In other embodiments, the polymer can include ≧80% mole percent of the recurring units Formula (III) and Formula (IV) based on ratio of total moles of recurring units of Formula (III) and recurring units of Formula (IV) to the total moles of recurring units in the polymer. In still other embodiments, the polymer can include ≧50% mole percent of the recurring units Formula (III) and Formula (IV) based on ratio of total moles of recurring units of Formula (III) and recurring units of Formula (IV) to the total moles of recurring units in the polymer. In yet still other embodiments, the polymer can include ≧30% mole percent of the recurring units Formula (III) and Formula (IV) based on ratio of total moles of recurring units of Formula (III) and recurring units of Formula (IV) to the total moles of recurring units in the polymer.

In some embodiments, the polymer can be a homopolymer. In other embodiments, the polymer can be a co-polymer. For example, in some embodiments, the co-polymer can include recurring units of both Formula (III) and Formula (IV).

In some embodiments, R² can be a first compound that comprises an agent. In other embodiments, R² can be hydrogen. In still other embodiments, R² can be C₁₋₁₀ alkyl. In yet still other embodiments, R² can be C₆₋₂₀ aryl. In some embodiments, R² can be ammonium. In other embodiments, R² can be alkali metal. In yet other embodiments, R² can be a polydentate ligand or a polydentate ligand precursor with protected oxygen atoms.

In some embodiments, at least one of R³ and R⁴ can be a first compound that comprises an agent. In other embodiments, at least one of R³ and R⁴ can be hydrogen. In still other embodiments, at least one of R³ and R⁴ can be C₁₋₁₀ alkyl. In yet still other embodiments, at least one of R³ and R⁴ can be C₆₋₂₀ aryl. In some embodiments, at least one of R³ and R⁴ can be ammonium. In other embodiments, at least one of R³ and R⁴ can be alkali metal. In still other embodiments, at least one of R³ and R⁴ can be a polydentate ligand or a polydentate ligand precursor with protected oxygen atoms. In some embodiments, both R³ and R⁴ can be an alkali metal.

In some embodiments, each R⁶ can independently be a second compound that comprises an agent. In other embodiments, each R⁶ can independently be hydrogen. In other embodiments, each R⁶ can independently be C₁₋₁₀ alkyl. In yet still other embodiments, each R⁶ can independently be C₆₋₂₀ aryl. In some embodiments, each R⁶ can independently be ammonium. In other embodiments, each R⁶ can independently be alkali metal. In yet other embodiments, each R⁶ can independently be a polydentate ligand or a polydentate ligand precursor with protected oxygen atoms.

In some embodiments, at least one of R⁷ and R⁸ can independently be a second compound that comprises an agent. In other embodiments, at least one of R⁷ and R⁸ can independently be hydrogen. In still other embodiments, at least one of R⁷ and R⁸ can be C₁₋₁₀ alkyl. In yet still other embodiments, at least one of R⁷ and R⁸ can independently be C₆₋₂₀ aryl. In some embodiments, at least one of R⁷ and R⁸ can independently be ammonium. In other embodiments, at least one of R⁷ and R⁸ can independently be alkali metal. In still other embodiments, at least one of R⁷ and R⁸ can independently be a polydentate ligand or a polydentate ligand precursor with protected oxygen atoms. In some embodiments, both R⁷ and R⁸ can be an alkali metal. Suitable alkali metals are known to those skilled in the art. In some embodiments, the alkali metal can be sodium.

As to the end-cap, any suitable optionally substituted C₁₋₁₀ alkyls and optionally substituted C₆₋₂₀ aryls can be utilized. In some embodiments, le can be an optionally substituted C₁₋₁₀ alkyl. In other embodiments, le can be an optionally substituted C₁₋₆ alkyl. In still other embodiments, le can be an unsubstituted C₁₋₆ alkyl. In yet still other embodiments, le can be methyl. In some embodiments, le can be an optionally substituted C₆₋₂₀ aryl. In other embodiments, le can be an optionally substituted C₆ aryl. In still other embodiments, le can be an unsubstituted C₆ aryl. In some embodiments, R⁵ can be an optionally substituted C₁₋₁₀ alkyl. In other embodiments, R⁵ can be an optionally substituted C₁₋₆ alkyl. In still other embodiments, R⁵ can be an unsubstituted C₁₋₆ alkyl. In yet still other embodiments, R⁵ can be methyl. In some embodiments, R⁵ can be an optionally substituted C₆₋₂₀ aryl. In other embodiments, R⁵ can be an optionally substituted C₆ aryl. In still other embodiments, R⁵ can be an unsubstituted C₆ aryl.

In some embodiments, the agent of the first compound that comprises an agent and the agent of the second compound that comprises an agent can be the same. In other embodiments, the agent of the first compound that comprises an agent and the agent of the second compound that comprises an agent can be the different.

Many types of agents may be used in the first compound that comprises an agent and the second compound that comprises an agent. The agent of the first compound that comprises an agent and the second compound that comprises an agent may comprise any type of active compound. For example, the agent(s) may be selected from a targeting agent, an optical imaging agent, a magnetic resonance imaging agent and a stabilizing agent.

In some embodiments, the agent of the first compound can be a drug. In some embodiments, the agent of the second compound can be a drug. Many different types of drugs may be used. In preferred embodiments, the drug can be an anticancer drug. In some embodiments, the anticancer drug can be a taxane. A non-limiting list of taxanes are paclitaxel and docetaxel. In some embodiments, when the anticancer drug is paclitaxel, the paclitaxel can be conjugated via the oxygen atom attached to the C2′-carbon. In some embodiments, when the anticancer drug is paclitaxel, the paclitaxel can be conjugated via the oxygen atom attached to the C7-carbon. In other embodiments, the anticancer drug can be a camptotheca, for example, camptothecin. In still other embodiments, the anticancer drug can be an anthracycline, such as doxorubicin. In yet still other embodiments, the anticancer drug can be selected from cisplatin (cDDP or cis-diamminedichloroplatinum(II)), carboplatin, oxaliplatin, and combinations thereof.

In some embodiments, the agent of the first compound can be an optical imaging agent. In some embodiments, the agent of the second compound can be an optical imaging agent. Examples of suitable optical imaging agents include, but are not limited to, acridine dye, a coumarine dye, a rhodamine dye, a xanthene dye, a cyanine dye, and a pyrene dye. For instance, specific optical imaging agents may include Texas Red, Alexa Fluor® dye, BODIPY® dye, Fluorescein, Oregon Green® dye, and Rhodamine Green™ dye, which are commercially available or readily prepared by methods known to those skilled in the art.

In some embodiments, the agent of the first compound can be a targeting agent. In some embodiments, the agent of the second compound can be a targeting agent. Many different types of targeting agents may be used. In some embodiments, the targeting agent can be selected from an arginine-glycine-aspartate (RGD) peptide, fibronectin, folate, galactose, an apolipoprotein, insulin, transferrin, a fibroblast growth factor (FGF), an epidermal growth factor (EGF), and an antibody. In some embodiments, the targeting agent can interact with a receptor selected from αᵥ,β₃-integrin, folate, asialoglycoprotein, a low-density lipoprotein (LDL), an insulin receptor, a transferrin receptor, a fibroblast growth factor (FGF) receptor, an epidermal growth factor (EGF) receptor, and an antibody receptor. In an embodiment, the arginine-glycine-aspartate (RGD) peptide can be cyclic(fKRGD).

In some embodiments, the agent of the first compound can be a magnetic resonance imaging agent. In some embodiments, the agent of the second compound can be a magnetic resonance imaging agent. In some embodiment, the magnetic resonance imaging agent can include a paramagnetic metal compound, such as a Gd(III) compound. Examples of suitable Gd(III) compounds include, but are not limited to, the following:

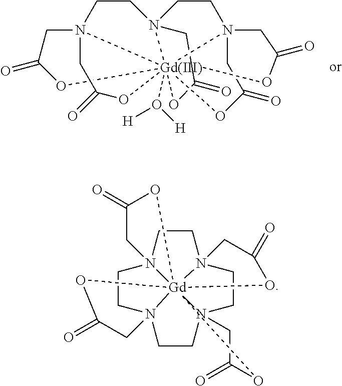

In some embodiments, the agent of the first compound can be a stabilizing agent. In some embodiments, the agent of the second compound can be a stabilizing agent. In preferred embodiments, the stabilizing agent can be polyethylene glycol (PEG).

Many types of polydentate ligand may be incorporated into a polymer described herein. In some embodiments, R² can be

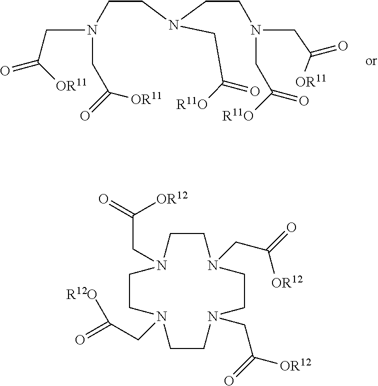

wherein each R^{H} can be independently hydrogen, ammonium, or an alkali metal; and wherein each R¹² can be independently hydrogen, ammonium, or an alkali metal. In some embodiments, at least one of R³ and R⁴ can be

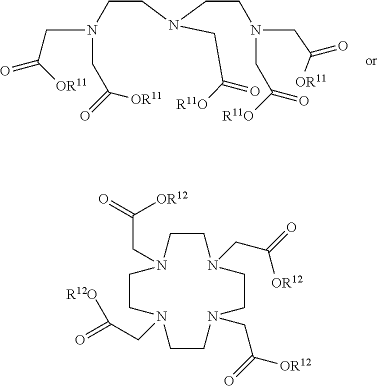

wherein each R^{H} can be independently hydrogen, ammonium, or an alkali metal; and wherein each R¹² can be independently hydrogen, ammonium, or an alkali metal. In some embodiments, R² and at least one of R³ and R⁴ can be a polydentate ligand, such as those described herein. When R² and at least one of R³ and R⁴ is a polydentate ligand, in some embodiments, R² and at least one of R³ and R⁴ can be the same. When R² and at least one of R³ and R⁴ is a polydentate ligand, in some embodiments, R² and at least one of R³ and R⁴ can be the different.

In some embodiments, R⁶ can be

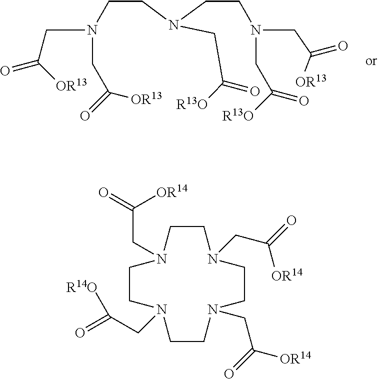

wherein each R¹³ can be independently hydrogen, ammonium, or an alkali metal; and wherein each R¹⁴ can be independently hydrogen, ammonium, or an alkali metal. In some embodiments, at least one of R⁶ and R⁷ can be

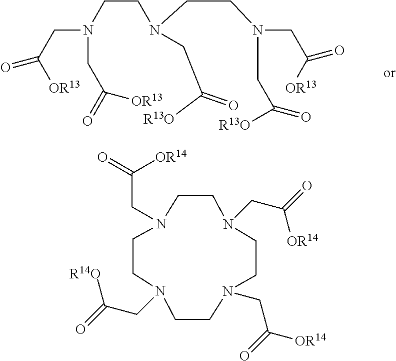

wherein each R¹³ can be independently hydrogen, ammonium, or an alkali metal; and wherein each R¹⁴ can be independently hydrogen, ammonium, or an alkali metal. In some embodiments, R⁶ and at least one of R⁷ and R⁸ can be a polydentate ligand, such as those described herein. When R⁶ and at least one of R⁷ and R⁸ is a polydentate ligand, in some embodiments, R⁶ and at least one of R⁷ and R⁸ can be the same. When R⁶ and at least one of R⁷ and R⁸ is a polydentate ligand, in some embodiments, R⁶ and at least one of R⁷ and R⁸ can be the different.

Various polydentate ligand precursors with protected oxygen atoms can be incorporated into a polymer described herein. In some embodiments, R² can be

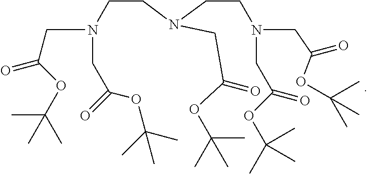

In some embodiments, at least one of R³ and R⁴ can be

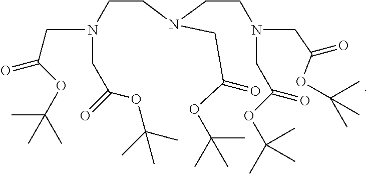

In some embodiments, R⁶ can be

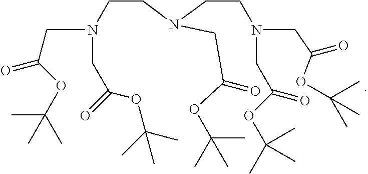

In some embodiments, at least one of R⁷ and R⁸ can be

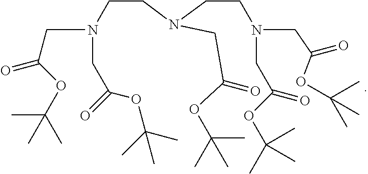

In some embodiments, a polymer described herein can include a drug and another agent that is not a drug. In other embodiments, a polymer described herein can include two drugs, wherein the drugs are not the same. For example, a polymer described herein could include a taxane and an anthracycline.

In some embodiments, the first compound that comprises an agent can further include a linker group. In some embodiments, the second compound that comprises an agent can further include a linker group. Suitable agents that can be included in the first compound and the second compound are described herein (e.g., drug, a targeting agent, an optical imaging agent, a magnetic resonance imaging agent, and a stabilizing agent). In some embodiments, the polydentate ligand can further include a linker group. In some embodiments, the polydentate ligand precursors with protected oxygen atoms can further include a linker group.

A linker group is a group that attaches, for example, the agent (or a compound that comprises an agent) to the polymer. In some embodiments, one or more of the aforementioned compounds can be attached to the polymer, e.g., to a recurring unit of Formulae (I), (II), (III), and/or (IV) through a linker group. The linker group may be relatively small. For instance, the linker group may comprise an amine, an amide, an ether, an ester, a hydroxyl group, a carbonyl group, or a thiol ether group. Alternatively, the linker group may be relatively large. For instance, the linker group may comprise an alkyl group, an ether group, an aryl group, an aryl(C₁₋₆ alkyl) group (e.g., phenyl-(CH₂)₁₋₄—), a heteroaryl group, or a heteroaryl(C₁₋₆ alkyl) group. In one embodiment, the linker can be —NH(CH₂)₁₋₄—NH—. In another embodiment, the linker can be —(CH₂)₁₋₄-aryl-NH—. The linker group can be attached to one or more of a first compound that comprises an agent, a second compound that comprises an agent, a polydentate ligand or a polydentate ligand precursors with protected oxygen atoms at any suitable position. For example, the linker group can be attached in place of a hydrogen at a carbon of one of the aforementioned compounds. The linker group can be added to the compounds using methods known to those skilled in the art.

The total amount of the agent may vary over a wide range. In some embodiments, the polymer can include a total amount of agent in the range of about 1% to about 99% (weight/weight) based on the mass ratio of the total amount of agent to the polymer. In other embodiments, the polymer can include a total amount of agent in the range of about 1% to about 70% (weight/weight) based on the mass ratio of the total amount of agent to the polymer. In still other embodiments, the polymer can include a total amount of agent in the range of about 1% to about 50% (weight/weight) based on the mass ratio of the total amount of agent to the polymer. In yet still embodiments, the polymer can include a total amount of agent in the range of about 20% to about 40% (weight/weight) based on the mass ratio of the total amount of agent to the polymer. In some embodiments, the polymer can include a total amount of agent in the range of about 30% to about 40% (weight/weight) based on the mass ratio of the total amount of agent to the polymer. In other embodiments, the polymer can include a total amount of agent of about 35% (weight/weight) based on the mass ratio of the total amount of agent to the polymer.

Additionally, the amount of a polydentate ligand or a polydentate ligand precursor with protected oxygen atoms present in the polymer can vary over a wide range. In some embodiments, the polymer can include a total amount of a polydentate ligand or a polydentate ligand precursor with protected oxygen atoms in the range of about 1% to about 99% (weight/weight) based on the mass ratio of the total amount of a polydentate ligand or a polydentate ligand precursor with protected oxygen atoms to the polymer. In other embodiments, the polymer can include a total amount of a polydentate ligand or a polydentate ligand precursor with protected oxygen atoms in the range of about 1% to about 70% (weight/weight) based on the mass ratio of the total amount of a polydentate ligand or a polydentate ligand precursor with protected oxygen atoms to the polymer. In still other embodiments, the polymer can include a total amount of a polydentate ligand or a polydentate ligand precursor with protected oxygen atoms in the range of about 1% to about 50% (weight/weight) based on the mass ratio of the total amount of a polydentate ligand or a polydentate ligand precursor with protected oxygen atoms to the polymer. In yet still embodiments, the polymer can include a total amount of a polydentate ligand or a polydentate ligand precursor with protected oxygen atoms in the range of about 20% to about 40% (weight/weight) based on the mass ratio of the total amount of a polydentate ligand or a polydentate ligand precursor with protected oxygen atoms to the polymer. In some embodiments, the polymer can include a total amount of a polydentate ligand or a polydentate ligand precursor with protected oxygen atoms in the range of about 30% to about 40% (weight/weight) based on the mass ratio of the total amount of a polydentate ligand or a polydentate ligand precursor with protected oxygen atoms to the polymer.

In some embodiments, the amounts of the agent(s), the percentage of the recurring unit of the Formulae (III) and/or (IV) in the polymer can be selected to provide a polymer solubility that is greater than that of a comparable polyglutamic acid that comprises substantially the same amount of the agent(s). The range of pH values over which the polymer comprising recurring units of the Formulae (III) and/or (IV), has greater solubility than that of a comparable polyglutamic acid may be narrow or broad. Solubility is measured by forming a polymer solution comprising at least 5 mg/mL of the polymer in 0.9 wt. % aqueous NaCl at about 22° C., and determining the optical clarity. In some embodiments, the polymer is soluble over a pH range of at least about three pH units. In another embodiment, the polymer is soluble over a pH range of at least about 8 pH units. In another embodiment, the polymer is soluble over a pH range of at least about 9 pH units. In another embodiment, the pH range over which the polymer is soluble includes at least one pH value in the range of about 2 to about 5, e.g., at pH=2, pH=3, pH=4 and/or pH=5. Preferably, the pH range over which the polymer is soluble is broader than the pH range over which the comparable polyglutamic acid is soluble. For example, in some embodiments, the polymer is soluble over a pH range that is at least about one pH unit broader, preferably at least about two pH units broader, than the pH range over which the comparable polyglutamic acid is soluble.

The amount of polymer placed in solution to measure solubility can also vary greatly. In some embodiments, solubility is measured when the tested polymer solution comprises at least about 5 mg/mL of the polymer. In another embodiment, solubility is measured when the tested polymer solution comprises at least about 10 mg/mL of the polymer. In another embodiment, solubility is measured when the tested polymer solution comprises at least about 25 mg/mL of the polymer. In another embodiment, solubility is measured when the tested polymer solution comprises at least about 100 mg/mL of the polymer. In another embodiment, solubility is measured when the tested polymer solution comprises at least about 150 mg/mL of the polymer. Those skilled in the art will understand that the comparable polyglutamic acid is tested at about the same concentration as that of the tested polymer.

The polymers described herein (for example, a polymer that can include a recurring unit of Formula (III) and/or Formula (IV) and an end-cap of Formula (I) or Formula (II)) may be prepared in various ways. In some embodiments, a polymeric reactant can be dissolved or partially dissolved in a solvent to form a dissolved or partially dissolved polymeric reactant. The polymeric reactant may comprise any suitable material capable of forming a polymer described herein. The dissolved or partially dissolved polymeric reactant can be then reacted with a second reactant, wherein the second reactant forms an end-cap.

Some embodiments described herein relate to a method of making a polymer that can include an end-cap selected Formula (I) and (II) and a recurring unit of selected from Formula (III) and Formula (IV) that can include dissolving or partially dissolving a polymeric reactant that can include at least one recurring unit selected from Formula (V) and Formula (VI) in a solvent to form a dissolved or partially dissolved polymeric reactant;

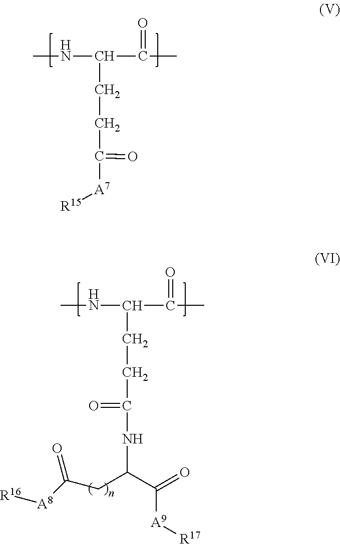

wherein o can be independently 1 or 2; A⁷, A⁸, and A⁹ can be oxygen; and R¹⁵, R¹⁶ and R¹⁷ can be independently selected from hydrogen, ammonium, and an alkali metal; and reacting the dissolved or partially dissolved polymeric reactant with a second reactant, wherein the second reactant can include a compound of Formula (VII):

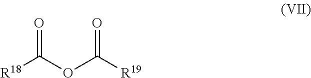

wherein R¹⁸ and R¹⁹ can be independently selected from an optionally substituted C₁₋₁₀ alkyl and an optionally substituted C₆₋₂₀ aryl.

Other embodiments described herein relate to a method of making a polymer that can include an end-cap selected Formula (I) and (II) and a recurring unit of selected from Formula (III) and Formula (IV) that can include dissolving or partially dissolving a polymeric reactant that can include a recurring unit of Formula (V) in a solvent to form a dissolved or partially dissolved polymeric reactant; reacting the dissolved or partially dissolved polymeric reactant with a second reactant, wherein the second reactant can include a compound of Formula (VII); and reacting the dissolved or partially dissolved polymeric reactant with an amino acid or an amino acid with protected oxygen atoms. In some embodiments, the amino acid can be selected from glutamic acid and aspartic acid. In other embodiments, the amino acid with protected oxygen atoms can be selected from glutamic acid and aspartic acid in which the single-bonded oxygen atoms are protected with one or more C₁₋₆ alkyl groups. A suitable example of an amino acid with protected oxygen atoms is

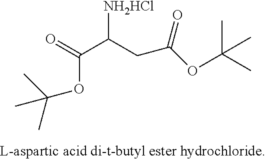

As to Formula (VII), in some embodiments, at least one R¹⁸ and R¹⁹ can be an optionally substituted C₁₋₁₀ alkyl. In other embodiments, at least one R¹⁸ and R¹⁹ can be an optionally substituted C₁₋₆ alkyl. In still other embodiments, at least one R¹⁸ and R¹⁹ can be an unsubstituted C₁₋₆ alkyl. In yet still other embodiments, at least one R¹⁸ and R¹⁹ can be methyl. In some embodiments, at least one R¹⁸ and R¹⁹ can be an optionally substituted C₆₋₂₀ aryl. In other embodiments, at least one R¹⁸ and R¹⁹ can be an optionally substituted C₆ aryl. In still other embodiments, at least one R¹⁸ and R¹⁹ can be an unsubstituted C₆ aryls. In some embodiments, both R¹⁸ and R¹⁹ can be an unsubstituted C₁₋₆ alkyl, such as methyl. In some embodiments, the second reactant can be acetic anhydride.

In some embodiments, a polymer reactant comprising a recurring unit of the Formula (III) can be produced starting with polyglutamic acid. Alternatively, in other embodiments, the polymer may be created by first converting the starting polyglutamic acid material into its salt form. The salt form of polyglutamic can be obtained by reacting polyglutamic acid with a suitable base, e.g., sodium bicarbonate. The weight average molecular weight of the polyglutamic acid is not limited, but is preferably from about 10,000 to about 500,000 daltons, and more preferably from about 25,000 to about 300,000 daltons.

In some embodiments, a polymer reactant comprising a recurring unit of the Formula (IV) can be produced starting with polyglutamic acid and an amino acid such as asparatic and/or glutamic acid. Alternatively, in other embodiments, the polymer may be created by first converting the starting polyglutamic acid material into its salt form. The salt form of polyglutamic can be obtained by reacting polyglutamic acid with a suitable base, e.g., sodium bicarbonate. An amino acid moiety can be attached to the pendant carboxylic acid group of the polyglumatic acid. The weight average molecular weight of the polyglutamic acid is not limited, but is preferably from about 10,000 to about 500,000 daltons, and more preferably from about 25,000 to about 300,000 daltons. Such a reaction may be used to create poly-(γ-L-aspartyl-glutamine) or poly-(γ-L-glutamyl-glutamine).

In some embodiments, the amino acid can be protected by one or more suitable protecting groups before attachment to the polyglutamic acid. One example of a protected amino acid moiety suitable for this reaction is L-aspartic acid di-t-butyl ester hydrochloride, shown below:

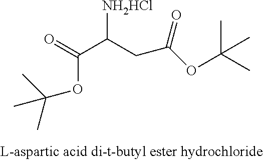

Reaction of the polyglutamic acid with the amino acid may take place in the presence of any suitable solvent. In some embodiments, the solvent can be an aprotic solvent. In a preferred embodiment, the solvent can be N,N′-dimethylformamide. In some embodiments, a coupling agent such as EDC, DCC, CDI, DSC, HATU, HBTU, HCTU, PyBOP®, PyBroP®, TBTU, and BOP can be used in the reaction between the polyglutamic acid and the amino acid. In some embodiments, polyglutamic acid and an amino acid can be reacted using a catalyst (e.g., DMAP).

The polymer may be recovered and/or purified by methods known to those skilled in the art. For example, the solvent may be removed by suitable methods, for instance, rotary evaporation. Additionally, the reaction mixture may be filtered into an acidic water solution to induce precipitation. The resultant precipitate can then be filtered, and washed with water.

In some embodiments, a polymer reactant comprising a recurring unit of the Formula (III) can also include a recurring unit of Formula (IV). One method for forming a polymer reactant comprising a recurring unit of the Formula (III) and a recurring unit of Formula (IV) is by starting with polyglutamic acid and reacting it with an amino acid such as asparatic and/or glutamic acid, in an amount that is less than 1.0 equivalents of the amino acid based on polyglutamic acid. For example, in one embodiment, 0.7 equivalents of an amino acid based on the polyglutamic acid can be reacted with polyglutamic acid, so that about 70% of the recurring units of the resulting polymer include the amino acid. As discussed above, the oxygen atoms of the amino acid can be protected using a suitable protecting group. In an embodiment, the amino acid may be L-aspartic acid or L-glutamic acid. In another embodiment, the oxygen atoms of the amino acid can be protected with t-butyl groups. If the oxygen atoms of the amino acid are protected, the protecting groups can be removed using known methods such as a suitable acid (e.g., trifluoroacetic acid).

In some embodiments, the dissolved or partially dissolved polymeric reactant can be reacted with a third reactant. In some embodiments, the third reactant can include a substituent selected from hydroxyl and amine.

In some embodiments, the dissolved or partially dissolved polymer reactant can be reacted with at least a portion of the second reactant before the dissolved or partially dissolved polymer reactant is reacted with the third reactant. In other embodiments, the dissolved or partially dissolved polymer reactant can be reacted with at least a portion of the second reactant at about the same time as the dissolved or partially dissolved polymer reactant is reacted with the third reactant. In still other embodiments, the dissolved or partially dissolved polymer reactant is reacted with at least a portion of the second reactant after reacting with the third reactant.

In some embodiments, the third reactant can be a compound that comprises an agent. Suitable agents are described herein. In some embodiments, the agent can be selected from a drug, a targeting agent, an optical imaging agent, a magnetic resonance imaging agent, and a stabilizing agent. In other embodiments, the third react can include be a group that comprises polydentate ligand or a polydentate ligand precursor with protected oxygen atoms.

In some embodiment, the third reactant can be a compound that comprises a drug. In some embodiments, the drug can be an anticancer drug, such as a taxane, a camptotheca or an anthracycline. In some embodiments, the taxane can be selected from paclitaxel and docetaxel. In some embodiments, the camptotheca can be camptothecin. In some embodiments, the anthracycline can be doxorubicin. In other embodiments, the anticancer drug can be selected from cisplatin (cDDP or cis-diamminedichloroplatinum(II)), carboplatin, oxaliplatin, and combinations thereof.

In some embodiment, the third reactant can be a compound that comprises a targeting agent. In some embodiments, the targeting agent can be selected from an arginine-glycine-aspartate (RGD) peptide, fibronectin, folate, galactose, an apolipoprotein, insulin, transferrin, a fibroblast growth factor (FGF), an epidermal growth factor (EGF), and an antibody. In other embodiments, the targeting agent can interact with a receptor selected from αᵥ,β₃-integrin, folate, asialoglycoprotein, a low-density lipoprotein (LDL), an insulin receptor, a transferrin receptor, a fibroblast growth factor (FGF) receptor, an epidermal growth factor (EGF) receptor and an antibody receptor.

In some embodiments, the third reactant can be a compound that comprises an optical imaging agent. In some embodiments, the optical imaging agent can be selected from an acridine dye, a coumarine dye, a rhodamine dye, a xanthene dye, a cyanine dye and a pyrene dye.

In some embodiments, the third reactant can be a compound that comprises magnetic resonance imaging agent. In some embodiments, the magnetic resonance imaging agent can include a Gd(III) compound. In some embodiments, the Gd(III) compound can include:

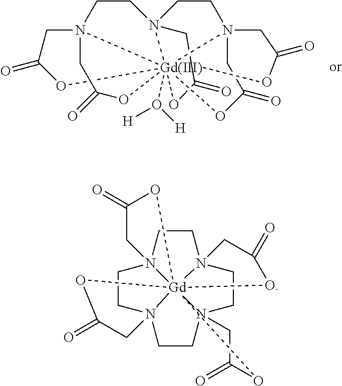

In some embodiments, the third reactant can be a compound that comprises a polydentate ligand. Examples of polydentate ligands include the following:

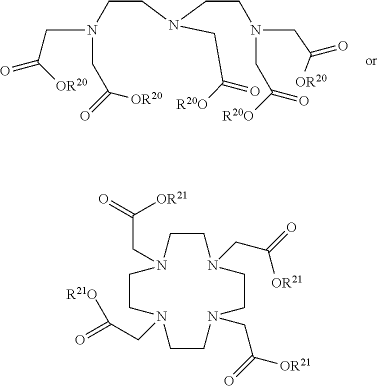

wherein each R²⁰ can be independently hydrogen, ammonium, or an alkali metal; and wherein each R²¹ can be independently hydrogen, ammonium, or an alkali metal.

In some embodiments, the third reactant can be a compound that comprises a polydentate ligand precursor with protected oxygen atoms. One example of a polydentate ligand precursor with protected oxygen atoms is shown below.

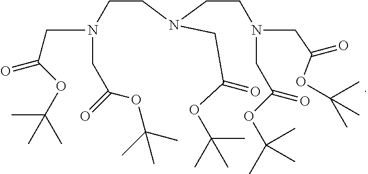

In some embodiments, the third reactant can be a compound that comprises a stabilizing agent, such as polyethylene glycol.

In some embodiments, method of making the polymer can include reacting the dissolved or partially dissolved polymeric reactant in the presence of a coupling agent. Any suitable coupling agent may be used. Examples of suitable coupling agents include, but are not limited to, 1-ethyl-3-(3-dimethylaminopropyl)-carbodiimide (EDC), 1,3-dicyclohexyl carbodiimide (DCC), 1,1′-carbonyl-diimidazole (CDI), N,N′-disuccinimidyl carbonate (DSC), N-[(dimethylamino)-1H-1,2,3-triazolo-[4,5-1)]pyridine-1-yl-methylene]-N-methylmethanaminium hexafluorophosphate N-oxide (HATU), 2-[(1H-benzotriazol-1-yl)-1,1,3,3-tetramethylaminium hexafluorophosphate (HBTU), 2-[(6-chloro-1H-benzotriazol-1-yl)-1,1,3,3-tetramethylaminium hexafluorophosphate (HCTU), benzotriazole-1-yl-oxy-tris-pyrrolidino-phosphonium hexafluorophosphate, bromo-tris-pyrrolidino-phosphonium hexafluorophosphate, 2-[(1H-benzotriazol-1-yl)-1,1,3,3-tetramethylaminium tetrafluoroborate (TBTU), and benzotriazol-1-yl-oxy-tris-(dimethylamino)phosphonium hexafluorophosphate (BOP).

Any suitable solvent that allows the reaction to take place may be used. In some embodiments, the solvent may be a polar aprotic solvent. In some embodiments, the solvent can be selected from N,N-dimethylformamide (DMF), dimethyl sulfoxide (DMSO), N-methyl-2-pyridone (NMP), and N,N-dimethylacetamide (DMAc).

In some embodiments, the reaction may further include reacting the dissolved or partially dissolved polymeric reactant in the presence of a catalyst. Any catalyst that promotes the reaction may be used. An example of a suitable catalyst is 4-dimethylaminopyridine (DMAP).

Conjugation of a compound that comprises a drug, a compound that comprises a targeting agent, a compound that comprises an optical imaging agent, a compound that comprises a magnetic resonance imaging agent, a compound that comprises a polydentate ligand, a compound that comprises a polydentate ligand precursor and/or a compound that comprises a stabilizing agent to the polymer acid or its salt form may be carried out in various ways, e.g., by covalently bonding the group comprising an agent, a polydentate ligand, and/or a polydentate ligand precursor with protected oxygen atoms to various polymers. One method for conjugating the aforementioned groups to the polymer is by using heat (e.g., heat from using a microwave method). Alternatively, conjugation may take place at room temperature. Appropriate solvents, coupling agents, catalysts, and/or buffers as generally known to those skilled in the art and/or as described herein may be used to form the polymer. As with polyglutamic acid, both the salt or acid form of the polymer obtained from polyglutamic acid and/or salt and an amino acid can be used as starting material for forming the polymer.

Suitable groups that can be conjugated to the polymers described herein include but are not limited to drugs, optical agents, targeting agents, magnetic resonance imaging agents (e.g., paramagnetic metal compounds), stabilizing agents, polydentate ligands, and polydentate ligand precursors with protected oxygen atoms.

As an example, in some embodiments, the polymer can be conjugated to an optical imaging agent such as those described herein. In an embodiment, the optical agent can be Texas Red-NH₂.

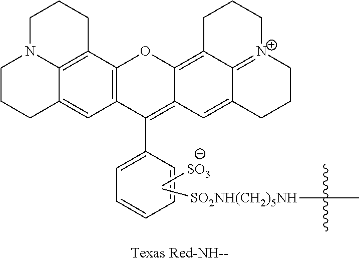

For example, a suitable polymeric reactant capable of forming a polymer comprising at least one end-cap selected from Formula (I) and/or Formula (II) and a recurring unit of the Formulae (III), and/or (IV) may be reacted with DCC, Texas Red-NH₂ dye, pyridine, and 4-dimethylaminopyridine. The mixture can be heated using a microwave method. In some embodiments, the reaction can be heated up to a temperature in the range of about 100° to about 150° C. In some embodiments, the time the materials can be heated ranges from about 5 to about 40 minutes. If desired, the reaction mixture can be cooled to room temperature. Suitable methods known to those skilled in the art can be used to isolate and/or purify the polymer. For instance, the reaction mixture can be filtered into an acidic water solution. Any precipitate that forms can then be filtered and washed with water. Optionally, the precipitate can be purified by any suitable method. For example, the precipitate can be transferred into acetone and dissolved, and the resulting solution can be filtered again into a sodium bicarbonate solution. If desired, the resulting reaction solution can be dialyzed in water using a cellulose membrane and the polymer can be lyophilized and isolated.

In some embodiments, a suitable polymeric reactant capable of forming a polymer comprising an end-cap selected from Formula (I) and/or Formula (II) and a recurring unit of the Formulae (III), and/or (IV) can be conjugated to a drug. Examples of suitable drugs are described herein. When the anti-cancer drug is paclitaxel, the paclitaxel may be joined to the polymer via the C2′-oxygen atom or the C7-oxygen atom. In some embodiments, the polymer can include both C2′-conjugated paclitaxel groups and C7-conjugated paclitaxel groups. The drug can be conjugated to the suitable polymeric reactant using the methods described above with respect to Texas-Red. In some embodiments, paclitaxel, preferably in the presence of a coupling agent (e.g, EDC and/or DCC) and a catalyst (e.g, DMAP), can be reacted with a suitable polymeric reactant capable of forming a polymer comprising an end-cap selected from Formula (I) and/or Formula (II) and a recurring unit of the Formulae (III), and/or (IV) in a solvent (e.g, an aprotic solvent such as DMF). Additional agents, such as pyridine or hydroxybenzotriazole may be used. In some embodiments, the reaction may take place over the period of 0.5-2 days. Suitable methods known to those skilled in the art can be used to isolate and/or purify the polymer. For example, the reaction mixture can be poured into an acidic solution to form a precipitate. Any precipitate that forms can then be filtered and washed with water. Optionally, the precipitate can be purified by any suitable method. For example, the precipitate can be transferred into acetone and dissolved, and the resulting solution can be filtered again into a sodium bicarbonate solution. If desired, the resulting reaction solution can be dialyzed in water using a cellulose membrane and the polymer can be lyophilized and isolated. The content of paclitaxel in the resulting polymer may be determined by UV spectrometry.

In some embodiments, a compound that comprises a drug, a compound that comprises a targeting agent, a compound that comprises an optical imaging agent, a compound that comprises a magnetic resonance imaging agent, a compound that comprises a polydentate ligand, a compound that comprises a polydentate ligand precursor and/or a compound that comprises a stabilizing agent can be reacted with an amino acid such as glutamic and/or aspartic acid so as to couple one or more of the aforementioned groups (e.g., covalently bonded) to the amino acid. The amino acid-compound that is formed can then be reacted with polyglutamic acid or its salt to form one of a polymer described herein. In some embodiments, paclitaxel can be reacted with glutamic acid to form a compound in which the paclitaxel is covalently bonded to the pendant carboxylic acid group of the glutamic acid. The glutamic acid-paclitaxel compound can then be reacted with polyglutamic acid or its salt to form one of the polymers described herein. In some embodiments, paclitaxel can be reacted with aspartic acid to form a compound in which the paclitaxel is covalently bonded to the pendant carboxylic acid group of the aspartic acid. The aspartic acid-paclitaxel compound can then be reacted with polyglutamic acid or its salt to form the polymer. If desired, the paclitaxel coupled to the amino acid by the C2′-oxygen can be separated from the paclitaxel coupled to the amino acid by the C7-oxygen using known separation methods (e.g., HPLC).

After formation of the polymer, any free amount of agent not covalently bonded to the polymer may also be measured. For example, thin layer chromatography (TLC) may be used to confirm the substantial absence of free paclitaxel remaining in the compositions of polymers conjugated to paclitaxel.

In some embodiments, a suitable polymeric reactant capable of forming a polymer comprising an end-cap selected from Formula (I) and/or Formula (II) and a recurring unit of the Formulae (III), and/or (IV) can be conjugated to a polydentate ligand. Suitable polydentate ligands include but are not limited to diethylenetriaminepentacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE),2,4-pentanedione (acac), and ethanedioate (ox). Appropriate solvents, coupling agents, catalysts, and/or buffers as generally known to those skilled in the art and/or described herein may be used to form the polymer. In other embodiments, the polymer comprising an end-cap selected from Formula (I) and/or Formula (II) and a recurring unit of the Formulae (III), and/or (IV) can be conjugated to a polydentate ligand precursor with protected oxygen atoms. As with polyglutamic acid, both the salt or acid form of the polymer obtained from polyglutamic acid and/or salt and an amino acid can be used as starting material for forming the polymer.

In some embodiments, the polydentate ligand can be DTPA. In other embodiments, the polydentate ligand can be DOTA. In some embodiments, the polydentate ligand such as DTPA (with or without protected oxygen atoms), preferably in the presence of a coupling agent (e.g., DCC) and a catalyst (e.g., DMAP), can be reacted in a solvent (e.g, an aprotic solvent such as DMF). If protecting groups are present, removal can achieved using suitable methods. For example, the polymer with the polydentate ligand precursor with protected oxygen atoms such as DTPA with oxygen atoms protected by t-butyl groups can be treated with acid such as trifluoroacetic acid. After removal of the protecting groups, the acid can be removed by rotary evaporation. In an embodiment, DTPA can be treated with a suitable base to remove the hydrogen atoms on the carboxylic acid —OH groups. In some embodiments, the base can be sodium bicarbonate.

In some embodiments, a suitable polymer reactant capable of forming a polymer comprising an end-cap selected from Formula (I) and/or Formula (II) and a recurring unit of the Formulae (III), and/or (IV) can be conjugated to a targeting agent. Exemplary targeting agents include, but are not limited to, arginine-glycine-aspartate (RGD) peptides, fibronectin, folate, galactose, apolipoprotein, insulin, transferrin, fibroblast growth factors (FGF), epidermal growth factors (EGF), and antibodies. Targeting agents can be chosen such that they interact with particular receptors. For example, a targeting agent can be chosen so that it interacts with one or more of the following receptors: αᵥ,β₃-integrin, folate, asialoglycoprotein, a low-density lipoprotein (LDL), an insulin receptor, a transferrin receptor, a fibroblast growth factor (FGF) receptor, an epidermal growth factor (EGF) receptor, and an antibody receptor. In an embodiment, the arginine-glycine-aspartate (RGD) peptide can be cyclic(fKRGD).

Both the salt or acid form of the polymeric reactant capable of forming a polymer comprising an end-cap selected from Formula (I) and/or Formula (II) and a recurring unit of the Formulae (III), and/or (IV) can be used as starting material for forming a polymer with a targeting agent. In some embodiments, the targeting agent preferably in the presence of a coupling agent (e.g., DCC) and a catalyst (e.g., DMAP), can be reacted with the polymer obtained from polyglutamic acid and/or salt and an amino acid in a solvent (e.g., an aprotic solvent such as DMF). After formation of the polymer, any free amount of agent not covalently bonded to the polymer may also be measured. For example, thin layer chromatography (TLC) may be used to confirm the substantial absence of any free targeting agent. Suitable methods known to those skilled in the art can be used to isolate and/or purify the polymer (e.g., lypholization).

In some embodiments, a suitable polymeric reactant capable of forming a polymer comprising an end-cap selected from Formula (I) and/or Formula (II) and a recurring unit of the Formulae (III), and/or (IV) can be conjugated to a magnetic resonance imaging agent. In some embodiments, the magnetic resonance imaging agent can include a Gd(III) compound. One method for forming the magnetic resonance imaging agent is by reacting a paramagnetic metal with the polymer comprising a polydentate ligand. Suitable paramagnetic metals include but are not limited to Gd(III), Indium-111, and Yttrium-88. For example, a polymer comprising DTPA can be treated with Gd(III) in a buffer solution for a period of several hours. Suitable methods known to those skilled in the art can be used to isolate and/or purify the polymer. For instance, the resulting reaction solution can be dialyzed in water using a cellulose membrane and the polymer can be lyophilized and isolated. The amount of paramagnetic metal may be quantified by inductively coupled plasma-optical emission spectroscopy (ICP-OES) measurement.

In some embodiments, a suitable polymeric reactant capable of forming a polymer comprising an end-cap selected from Formula (I) and/or Formula (II) and a recurring unit of the Formulae (III), and/or (IV) can be conjugated to a stabilizing agent. In some embodiments, the stabilizing agent can be polyethylene glycol. In one method, the stabilizing agent, preferably in the presence of a coupling agent (e.g., DCC) and a catalyst (e.g., DMAP), can be reacted with the polymer obtained from polyglutamic acid and/or salt and an amino acid in a solvent (e.g., an aprotic solvent such as DMF). Progress of the reaction can be measured by any suitable method such as TLC. The resulting polymer can be purified using methods known to those skilled in the art such as dialysis.

Additional information regarding methods suitable polymeric reactants and methods for conjugating agents, polydentate ligands and polydentate ligand precusors with protected oxygens atoms are described in U.S. Pat. No. 5,977,163, filed Mar. 11, 1997; U.S. Patent Publication Nos. 2007-0128118, filed on Dec. 12, 2006; 2008-0181852, filed Jan. 24, 2008; 2008-0253969, filed Apr. 8, 2008; 2008-0279778, filed May 8, 2008; and 2008-0279782, filed May 8, 2008; where are all hereby incorporated by reference in their entireties.

### Pharmaceutical Compositions

Some embodiments described herein relate to a composition that can include one or more polymers described herein and at least one selected from a pharmaceutically acceptable excipient, a carrier, and a diluent. In some embodiments, prodrugs, metabolites, stereoisomers, hydrates, solvates, polymorphs, and pharmaceutically acceptable salts of the compounds disclosed herein (e.g., a polymer described herein) are provided.

A “prodrug” refers to an agent that is converted into the parent drug in vivo.

The term “pharmaceutical composition” refers to a mixture of a compound disclosed herein (e.g., a polymer described herein) with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” refers to a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” refers to chemical compounds diluted in water that will dissolve the compound of interest (e.g., a polymer described herein) as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound. The term “physiologically acceptable” refers to a carrier or diluent that does not abrogate the biological activity and properties of the compound.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like.

If the manufacture of pharmaceutical formulations involves intimate mixing of the pharmaceutical excipients and the active ingredient in its salt form, then it may be desirable to use pharmaceutical excipients which are non-basic, that is, either acidic or neutral excipients.

In various embodiments, the compounds disclosed herein (e.g., the polymers and/or the agent(s) that it comprises) can be used alone, in combination with other compounds disclosed herein, or in combination with one or more other agents active in the therapeutic areas described herein.

In some embodiments, the pharmaceutical composition can include one or more physiologically acceptable surface active agents, carriers, diluents, excipients, smoothing agents, suspension agents, film forming substances, and coating assistants, or a combination thereof; and a compound (e.g., a polymer described herein) disclosed herein. Acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

In various embodiments, the pharmaceutical compositions and polymers disclosed herein may be in the form of an injectable liquid.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

For transmucosal administration, penetrants appropriate to the barrier to be permeated may be used in the formulation.

Pharmaceutical formulations for parenteral administration, e.g., by bolus injection or continuous infusion, include aqueous solutions of the active compounds (e.g., a polymer disclosed herein) in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ.

The pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

### Methods of Administration

Some embodiments described herein relate to a method of treating or ameliorating a disease or condition that can include administering an effective amount of one or more of the polymers described herein or one or more of the pharmaceutical compositions described herein to a subject in need thereof. In some embodiments, the disease or condition can be selected from lung tumor, breast tumor, colon tumor, ovarian tumor, prostate tumor, and melanoma tumor. In some embodiments, the disease or condition can be selected from lung cancer, breast cancer, colon cancer, ovarian cancer, prostate cancer, and melanoma.

Other embodiments described herein relate to a method of diagnosing a disease or condition that can include administering an effective amount of one or more of the polymers described herein or one or more of the pharmaceutical compositions described herein to a subject in need thereof. In some embodiments, the disease or condition can be selected from lung tumor, breast tumor, colon tumor, ovarian tumor, prostate tumor, and melanoma tumor. In some embodiments, the disease or condition can be selected from lung cancer, breast cancer, colon cancer, ovarian cancer, prostate cancer, and melanoma.

Still other embodiments described herein relate to provides a method of imaging a portion of tissue that can include administering an effective amount of one or more of the polymers described herein or one or more of the pharmaceutical compositions described herein to a subject in need thereof. In some embodiments, the tissue can be from a tumor selected from lung tumor, breast tumor, colon tumor, and ovarian tumor.

In some embodiments, one or more of the polymers described herein or one or more of the pharmaceutical compositions described herein can be administered intravenously.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The term “effective amount” is used to indicate an amount of an active compound, or pharmaceutical agent, that elicits the biological or medicinal response indicated. For example, an effective amount of compound can be the amount needed to prevent, alleviate or ameliorate symptoms of disease or prolong the survival of the subject being treated This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the subject. In some embodiments, the compounds will be administered for a period of continuous therapy, for example for a week or more, or for months or years.

In instances where human dosages for compounds have been established for at least some condition, those same dosages my be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

The following examples are provided for the purposes of further describing the embodiments described herein, and do not limit the scope of the claims.

### General

1,3-dicyclohexyl carbodiimide (DCC), N-(3-dimethylaminopropyl)-N′-ethylcarbodiimide hydrochloride (EDC), hydroxybenzotriazole (HOBt), pyridine; 4-dimethylaminopyridine (DMAP), N,N′-dimethylformamide (DMF), chloroform, sodium bicarbonate, poly(L-glutamic acid) sodium salts (sPGA) was purchased from Sigma-Aldrich Chemical company. Poly(L-glutamic acid) sodium salt (PGA) was synthesized according to the procedure in literature (Murray Goodman and John Hutchison, “The Mechanisms of Polymerization of N-Unsubstituted N-Carboxyanhydrides.” JACS 1966, 88, 3627-3630. sPGA and PGA were converted into poly(L-glutamic acid) using a diluted solution of 2 N hydrochloric acid. Trifluoroacetic acid (TFA) was purchased from Bioscience. L-glutamic acid di-t-butyl ester hydrochloride (H-Glu(OtBu)-OtBu.HCl) was purchased from Novabiochem (La Jolla, Calif.). Paclitaxel was purchased from NuBlocks. Sulforhodamine B dye for cytotoxic MTT test (cell viability) was purchased from Molecular Imaging Products Company (Michigan). ¹H NMR was obtained from Joel (400 MHz).

### Example 1

### Capped sPGA 1

Poly(L-glutamic acid) sodium salt (average molecular weight of 20,000 daltons determined by gel chromatography with multi-angle light scattering (MALS) detector, 5.0 g) purchased from Sigma-Aldrich chemical company was dissolved in water (100 mL). An aliquot (100 μL) of the solution was withdrawn and tested with ninhydrin assay to confirm the amino terminal group (NH₂—) of the poly(L-glutamic acid) sodium salt. The test result showed that ninhydrin test was positive. A solution of capping agent, acetic anhydride (10 mL), in dimethylformamide (DMF, 10 mL) was added to the solution of poly(L-glutamic acid) sodium salt with vigorous stirring. After 10 minutes of stirring, another aliquot (100 μL) of the reaction mixture was withdrawn and tested with ninhydrin assay to confirm the disappearance of the amino terminal group (NH₂—) of the poly(L-glutamic acid) sodium salt. The test result showed that ninhydrin test was negative indicating the capping was completed. The solution was acidified with acetic acid (20 mL), and a precipitate formed. The solid was collected after centrifugation and re-dissolved with 1 N sodium bicarbonate solution (150 mL) for 24 hours. The capped poly(L-glutamic acid) sodium salt was dialyzed in semi-permeable cellulose membrane against water for 24 hours. The capped poly(L-glutamic acid) sodium salt was then lyophilized and characterized by gel chromatography with a MALS detector. The capped poly(L-glutamic acid) sodium was obtained (1.61 g, 32% yield, with average molecular weight of 20,000 Daltons).

### Example 2

### Capped PGA 2

Poly(L-glutamic acid) sodium salt (average molecular weight of 20,000 daltons determined by MALS, 5.0 g) synthesized according to a procedure in the literature (Murray Goodman and John Hutchison, “The Mechanisms of Polymerization of N-Unsubstituted N-Carboxyanhydrides.” JACS 1966, 88, 3627-3630). Capped PGA was prepared following the procedure described in Example 1, using the aforementioned literature prepared PGA as the starting material. The capped poly(L-glutamic acid) sodium was obtained (1.57 g, 31% yield, with average molecular weight of 20,000 Daltons).

### Example 3

### Capped PGA 2

Capped PGA was prepared following the procedure in Example 2, except acetone was used in place of DMF. The starting PGA was the literature prepared PGA as described in Example 2. The capped poly(L-glutamic acid) sodium was obtained. (Yield 63%).

### Example 4

### Non-Capped sPGA-PTX 3

Poly(L-glutamic acid) sodium salt (average molecular weight of 20,000 daltons determined by gel chromatography with multi-angle light scattering (MALS) detector) was purchased from Sigma-Aldrich chemical company and converted into an acid form poly(L-glutamic acid) (sPGA). sPGA (531 mg) was dissolved in DMF 5 mL) in a oven-dried 100-mL round bottom flask, equipped with a Teflon magnetic stir bar under an argon atmosphere. Paclitaxel (313 mg) and 4-dimethylaminopyridine (9 mg) were added. The reaction mixture was stirred for 30 minutes. A solution of 1,3-diisopropylcarbodiimide (74 μL, density of 0.815 g/mL) in DMF (2 mL) was added to the reaction mixture. The reaction was allowed to stir for 16 hours at room temperature. The reaction was cooled to 5° C. and 10% sodium chloride solution (43.7 mL) was added slowly to precipitate out the non-capped sPGA-PTX 3. The precipitate was collected after centrifugation at 6,000 rpm, and suspended in water (20 mL). A solution of 1 N sodium bicarbonate (15.2 mL) was added slowly with vigorous stirring until pH 7. The solution was then stirred for 1 hour, and filtered through 0.45 μm filter to remove impurities. The filtrate was cooled to 5° C. and slowly acidified with 1 N HCl solution until pH 3. The precipitated solid was collected after centrifugation at 6,000 rpm for 10 minutes. The wet solid was washed twice with water. The product was lyophilized to yield the non-capped sPGA-PTX 3 (200 mg, 24% yield).

### Example 5

### Capped sPGA-PTX 4

The capped poly(L-glutamic acid) sodium (average molecular weight of 20,000 Daltons determined by gel chromatography with multi-angle light scattering (MALS) detector), prepared according to the above described procedure of capped sPGA (Example 1), was converted into its acid form, capped poly(L-glutamic acid) (capped sPGA). Capped sPGA (2.13 g) was dissolved in DMF (22.5 mL) in an oven-dried 100-mL round bottom flask, equipped with a Teflon magnetic stir bar under an argon atmosphere. Paclitaxel (1.25 g) and 4-dimethylaminopyridine (0.034 g) were added. The reaction mixture was stirred for 30 minutes. A solution of 1,3-diisopropylcarbodiimide (0.31 mL, density of 0.815 g/mL) in DMF (6.25 mL) was added to the reaction mixture. The reaction was allowed to stir for 16 hours at room temperature. The reaction was cooled to 5° C. and 10% sodium chloride solution (43.7 mL) was added slowly to precipitate out the non-capped sPGA-PTX 4. The precipitate was collected after centrifugation at 6,000 rpm, and suspended in water (20 mL). A solution of 1 N sodium bicarbonate (15.2 mL) was added slowly with vigorous stirring until pH 7. The solution was stirred for 1 hour, and filtered through 0.45 μm filter to remove impurities. The filtrate was cooled to 5° C. and slowly acidified with 1 N HCl solution until pH 3. The precipitated solid was collected after centrifugation at 6,000 rpm for 10 minutes. The wet solid was washed twice with water. The product was lyophilized to yield the non-capped sPGA-PTX 4 (2.63 g, 78% yield).

### Example 6

### Capped PGA-PTX 5

The capped poly(L-glutamic acid) sodium (average molecular weight of 20,000 Daltons determined by gel chromatography with multi-angle light scattering (MALS) detector), prepared according to the above described procedure of capped PGA 2 (Example 2), was converted into an acid form capped poly(L-glutamic acid) (capped PGA). Capped PGA (600 mg) was dissolved in DMF (22.5 mL) in an oven-dried 100-mL round bottom flask, equipped with a Teflon magnetic stir bar under an argon atmosphere. Paclitaxel (340 mg) and 4-dimethylaminopyridine (10 mg) were added. The reaction mixture was stirred for 30 minutes. A solution of 1,3-diisopropylcarbodiimide (0.08 mL, density of 0.815 g/mL) in DMF (6.25 mL) was added to the reaction mixture. The reaction was allowed to stir for 16 hours at room temperature. The reaction was cooled to 5° C. and 10% sodium chloride solution (43.7 mL) was added slowly to precipitate out the non-capped sPGA-PTX 5. The precipitate was collected after centrifugation at 6,000 rpm, and suspended in water (20 mL). A solution of 1 N sodium bicarbonate (15.2 mL) was added slowly with vigorous stirring until pH 7. The solution was stirred for 1 hour, and filtered through 0.45 μm filter to remove impurities. The filtrate was cooled to 5° C. and slowly acidified with 1 N HCl solution until pH 3. The precipitated solid was collected after centrifugation at 6,000 rpm for 10 minutes. The wet solid was washed twice with water. The product was lyophilized to yield the non-capped sPGA-PTX 5 (0.55 g, 58% yield).

### Example 7

### Non-Capped sPGGA 6

Poly(L-glutamic acid) sodium salt (average molecular weight of 20,000 daltons determined by gel chromatography with multi-angle light scattering (MALS) detector) was purchased from Sigma-Aldrich chemical company. Poly(L-glutamic acid) sodium salt (1.0 g), EDC (3.7 g), HOBt (1.8 g), and H-glu(OtBu)-(OtBu)-HCl (3.7 g) were mixed in DMF (30 mL). The reaction mixture was stirred at room temperature for 15-24 hours, and then was poured into distilled water solution (200 mL). A white precipitate formed. The precipitate was filtered and washed with water. After freeze drying, the intermediate polymer was obtained (2.3 g). The structure of the intermediate polymer was confirmed via ¹H-NMR by the presence of a peak for the O-tBu group at 1.4 ppm.

The intermediate polymer was treated with TFA (20 mL) for 5-8 hours. The TFA was then partially removed by rotary evaporation. Water was added to the residue. The residue was then dialyzed using semi-membrane cellulose (molecular weight cut-off 10,000 daltons) in reverse-osmosis water (4 time water changes) overnight. Poly-(γ-L-glutamyl-glutamine) was colorless at pH 7 in water after dialysis. Non-capped sPGGA 6 was obtained (1.3 g) as white powder after being lyophilized. The polymer structure was confirmed via ¹H-NMR by the disappearance of the peak for the O-tBu group at 1.4 ppm.

### Example 8

### Capped PGGA 7

The capped poly(L-glutamic acid) sodium (average molecular weight of 20,000 Daltons determined by gel chromatography with multi-angle light scattering (MALS) detector), prepared according to the above described procedure of capped PGA 2 (Example 2). Capped poly(L-glutamic acid) sodium salt (1.0 g), EDC (3.7 g), HOBt (1.8 g), and H-glu(OtBu)-(OtBu)-HCl (3.7 g) were mixed in DMF (30 mL). The reaction mixture was stirred at room temperature for 15-24 hours, and then was poured into distilled water solution (200 mL). A white precipitate formed. The precipitate was filtered and washed with water. After freeze drying, The intermediate polymer was obtained (2.5 g). The intermediate polymer structure was confirmed via ¹H-NMR by the presence of a peak for the O-tBu group at 1.4 ppm.

The intermediate polymer was treated with TFA (20 mL) for 5-8 hours. The TFA was then partially removed by rotary evaporation. Water was added to the residue, and the residue was dialyzed using semi-membrane cellulose (molecular weight cut-off 10,000 daltons) in reverse-osmosis water (4 time water changes) overnight. Poly-(γ-L-glutamyl-glutamine) was transparent at pH 7 in water after dialysis. Capped sPGGA 7 was obtained (1.8 g) as white powder after being lyophized. The polymer structure was confirmed via ¹H-NMR by the disappearance of the peak for the O-tBu group at 1.4 ppm.

### Example 9

### Characterization of Polymers

Molecular weights of polymers were determined by size exclusion chromatography (SEC) combined with a multi-angle light scattering (MALS) (Wyatt Corporation) detector:

### SEC-MALS Analysis Conditions:

HPLC system: Agilent 1200
Column: Shodex SB 804 HQ
Mobile Phase: 50 mM PBS (pH 6.5) with 45% methanol, 200 ppm sodium azide,
Flow Rate: 0.7 ml/min
MALS detector: DAWN HELEOS from Wyatt
DRI detector: Optilab rEX from Wyatt
Software: ASTRA from Wyatt
Sample Concentration: 2 mg/ml
Injection volume: 100 μl

Dn/dc value of poly(L-glutamic acid) sodium salt, poly(L-glutamyl-glutamine acid), and poly(L-glutamic acid)-paclitaxel conjugate was 0.180, 0.145, and 0.173, respectively. BSA was used as a control before actual samples are run.

The content of paclitaxel in polymer-paclitaxel conjugates was estimated by UV/Vis spectrometry (Lambda Bio 40, PerkinElmer) based on a standard curve generated with known concentrations of paclitaxel in methanol (λ=228 nm).

FIG. 5 illustrates chromatograms and summary tables from the molecular weight experiments of non-capped sPGA-PTX 3, capped sPGA-PTX 4 and capped PGA-PTX 5. As shown in FIG. 5, the chromatogram of non-capped sPGA-PTX 3 shows a mixture of sPGA-PTX aggregate (Peak 1) and sPGA-PTX polymer (Peak 2). By comparison, the chromatogram of capped sPGA-PTX 4 shows less sPGA-PTX aggregate (Peak 1). Moreover, the amount of aggregation is less for PGA-PTX 4 compared to sPGA-PTX 3 as shown by the comparison of weight average molecular weights (PGA-PTX aggregate=2.894×10⁷; sPGA-PTX aggregate=9.152×10⁶). The chromatogram for capped PGA-PTX 5 shows undetectable amounts of polymer aggregate (absence of Peak 1). Thus, by capping the polymer, the amount of aggregation can be reduced. Moreover, by capping the polymer, the resulting polymer is less polydisperse, as shown by a comparison of the polydispersities (non-capped sPGA-PTX 3=5.321; capped sPGA-PTX 4=1.628 and capped PGA-PTX 5=1.350).

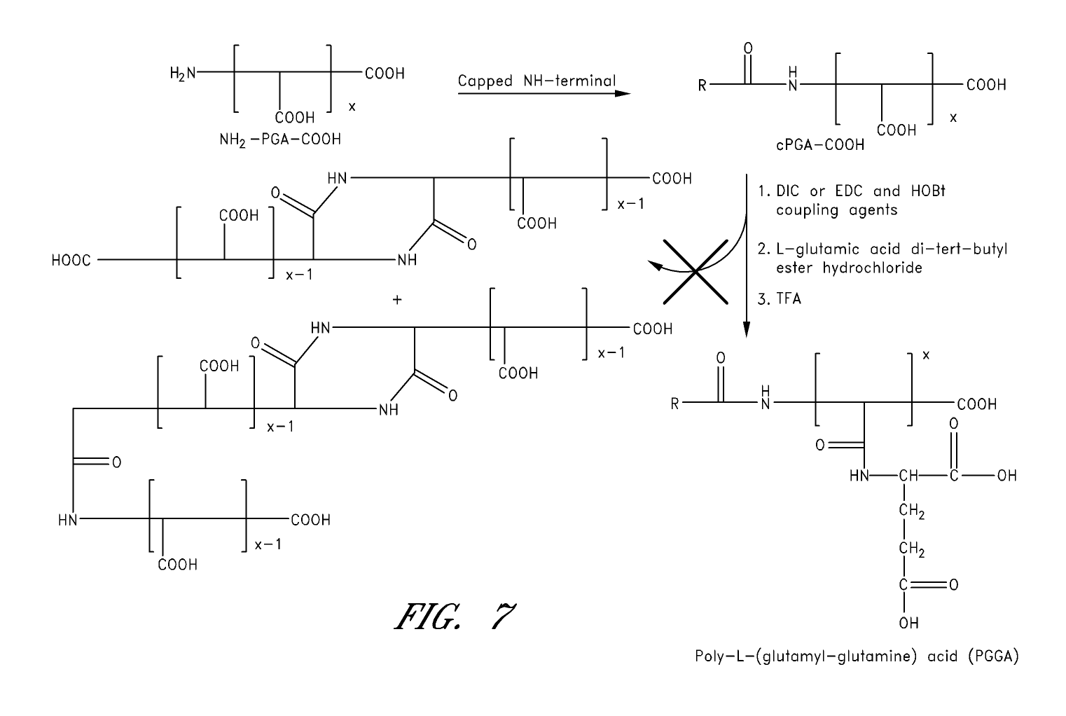

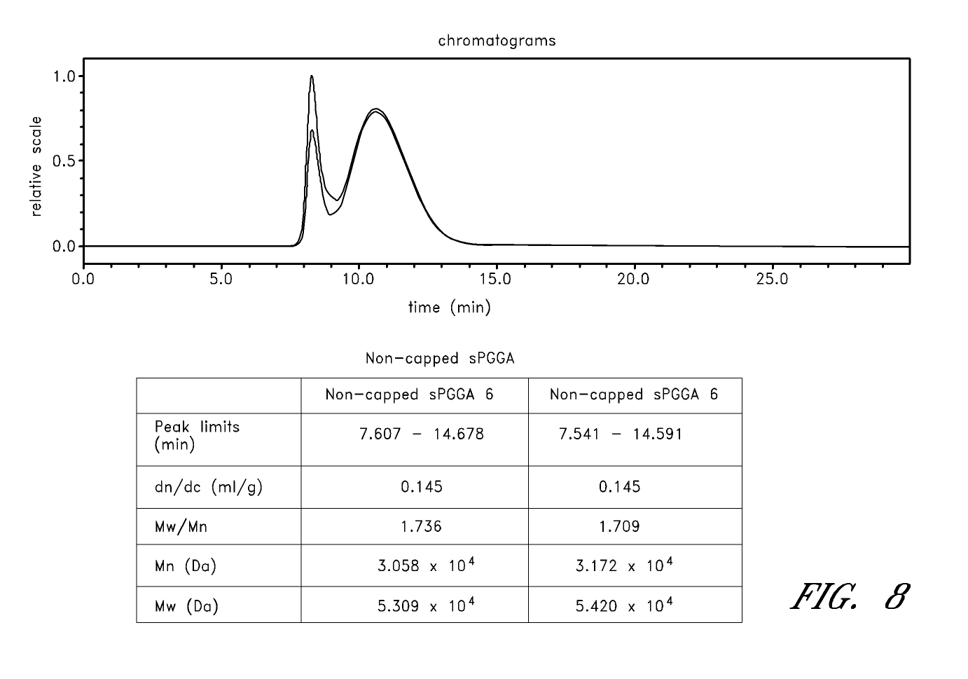

FIG. 8 illustrates chromatograms and summary tables from the molecular weight experiments of non-capped sPGGA 6. The molecular weight experiments were conducted with two different batches of non-capped sPGGA 6. As shown by FIG. 8, non-capped sPGGA 6 exhibits the presence of both sPGGA aggregate (peak at approximately 7.6 min) and sPGGA polymer (broader peak at approximately 10-14 min).

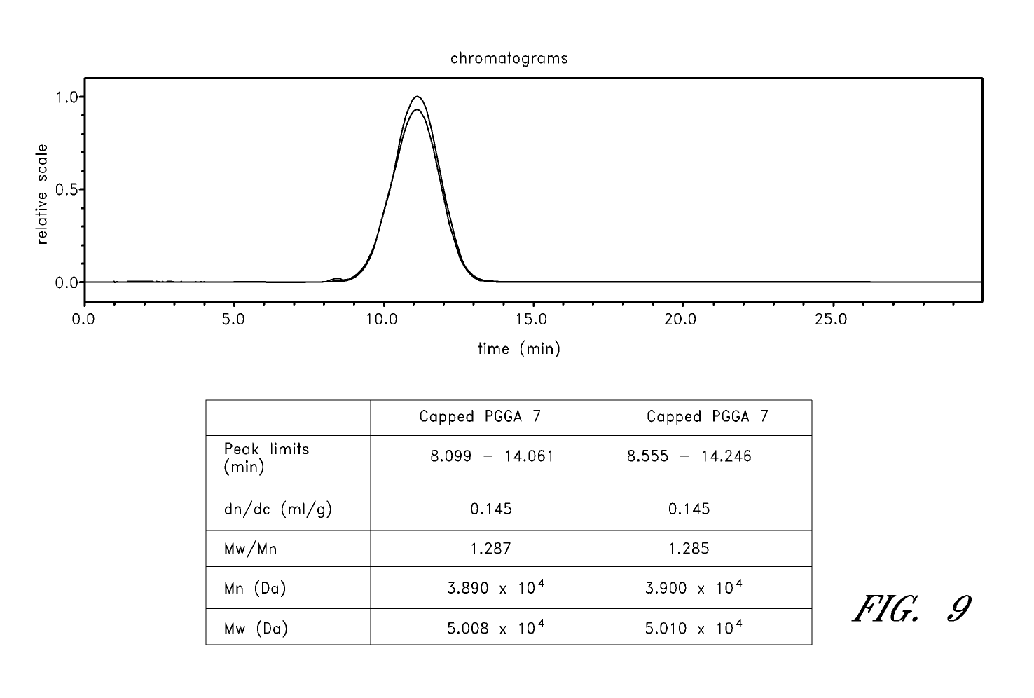

FIG. 9 illustrates chromatograms and summary tables from the molecular weight experiments of capped PGGA 7. The molecular weight experiments were conducted with two different batches of capped PGGA 7. As shown by FIG. 9, capped PGGA 7 exhibits only the presence of PGGA polymer. Any PGGA aggregate was undetectable. In addition, a comparison of the polydispersities demonstrates the capped polymer is less polydisperse compared to the non-capped polymer.

### Example 10

### Cell Culture and Preparation

B16F0 cells were purchased from ATCC(CRL-6322, ATCC American Type Culture Collection, Rockville, Md.) and were grown in Dulbecco's modified Eagle's medium (DMEM) with 10% fetal bovine serum and 100 units/mL penicillin. The cells were grown at 37° C. in 5% CO₂ environment. The culture medium was removed and discarded. The cells were rinsed with Dulbecco Phosphate Buffer Solution (DPBS), Trypsin-ethylenediaminetetra-acetic acid (EDTA) solution (0.5 ml) was added, and the cells were observed under an inverted microscope to make sure that they were dispersed. Complete growth medium (6.0 to 8.0 ml) was added, and the cells were aspirated by gently pipetting. The cell suspension in appropriate aliquots was transferred to new culture plates. The cells were allowed to grow at 37° C. in 5% CO₂ for 24 hours before further experiments.

### Example 11

### In Vitro Cytotoxicity MTT Studies

Polymer conjugates described herein containing an anti-cancer drug are evaluated for their effect on the proliferation of B16F0 melanoma cells at several different concentrations of the drug. Cytotoxic MTT assay is carried out as reported in Monks et al. JNCI 1991, 83, 757-766, which is hereby incorporated by reference in its entirety. Polymer conjugates are prepared as described in Examples 1-8.

### Example 12

### Binding Studies

The binding assays are carried out as described in Line et al, [Journal of Nuclear Medicine], 2005, 46, 1552-1560; and Mitra et al., [Journal of Controlled Release], 2006, 114, 175-183, both of which are hereby incorporated by reference in their entireties. Polymers conjugates described herein are prepared as described in Examples 1-8.

### Example 13

### Animals and Tumor Models

Nude mice (6-7 week old, body weight 25-30 g, male) are purchased from Charles River Lab (Willington, Mass.). B16 cell line is purchased from ATCC(CRL-6322, ATCC American Type Culture Collection, Rockville, Md.). The B16 cells are cultured in RMPI 1640 supplemented with 10% Fetal bovine serum, 2 μM Glutamine, 1 mM non-essential amino acids, 1 mM sodium pyruvate, 100 U/ml penicillin and 100 ug/ml streptomycin. The B16 cells harvested from tissue culture is counted and re-suspended to a concentration of 5×10⁶ per mL. Using a TB syringe, 0.2 mL (a total of 1×10⁶ cells) is administered via subcutaneous injection into each mouse. One tumor is inoculated per animal at the right hip. The site of tumor inoculation is shaved prior to inoculation to make it easier to measure the tumor as it grows.

### Example 14

### Magnetic Resonance Imaging for Tumor Accumulation

Images of mice is acquired on a GE 3T MR scanner using a knee coil pre- and post-contrast. The following imaging parameters are TE: minful, TR=250 ms, FOV: 8 and 24 slices/slab, and 1.0 mm coronal slice thickness. Polymer conjugates with a compound comprising a magnetic resonance imaging agent, such as Gd(III), and Omniscan-Gd(III)-(DTPA-BMA (0.1 mmol Gd(III)/kg), a control, are injected via a tail vein into anesthetized mice. The dose of injection of the polymer conjugate and Omniscan™ is 0.1 mmol Gd(III)/kg. Images are acquired at pre-injection and at 6 minutes to 4 hours post-injection of the contrast agents.

It will be understood by those of skill in the art that numerous and various modifications can be made without departing from the spirit of the present invention. Therefore, it should be clearly understood that the forms of the present invention are illustrative only and not intended to limit the scope of the present invention.

What is claimed is:

1. A polymer comprising an end-cap selected from the group consisting of Formula (I) and Formula (II); and at least one recurring unit selected from the group consisting of Formula (III) and Formula (IV): 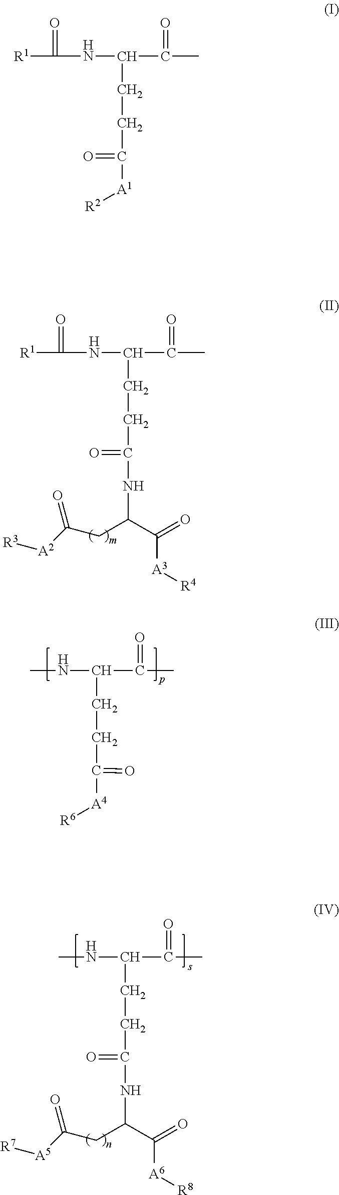
wherein:
m is independently 1 or 2;
R¹ and R⁵ are independently selected from the group consisting of an optionally substituted C₁₋₁₀ alkyl and an optionally substituted C₆₋₂₀ aryl;
A¹, A² and A³ are independently oxygen or NR⁹, wherein R⁹ is hydrogen or C₁₋₄ alkyl;
R², R³ and R⁴ are independently selected from the group consisting of H, C₁₋₁₀ alkyl, C₆₋₂₀ aryl, ammonium, alkali metal, a polydentate ligand, a polydentate ligand precursor with protected oxygen atoms and a first compound that comprises an agent;
n is independently 1 or 2;
p is 0 or an integer ≧1;
s is 0 or an integer ≧1;
provided that the sum of p+s is ≧1;
each A⁴, each A⁵ and each A⁶ are independently oxygen or NR¹⁰, wherein each R¹⁰ is hydrogen or C₁₋₄ alkyl; and
each R⁶, each R⁷ and each R⁸ are independently selected from the group consisting of H, C₁₋₁₀ alkyl, C₆₋₂₀ aryl, ammonium, alkali metal, a polydentate ligand, a polydentate ligand precursor with protected oxygen atoms and a second compound that comprises an agent; and
each agent is independently selected from the group consisting of a drug, a targeting agent, an optical imaging agent, a magnetic resonance imaging agent, and a stabilizing agent.

2. The polymer of claim 1, wherein the agent of the first compound is selected from the group consisting of a stabilizing agent, an optical imaging agent, an anticancer drug, a targeting agent, and a magnetic resonance imaging agent.

3. The polymer of claim 2, wherein the stabilizing agent is PEG.

4. The polymer of claim 2, wherein the optical imaging agent is selected from the group consisting of an acridine dye, a coumarine dye, a rhodamine dye, a xanthene dye, cyanine dye, and a pyrene dye.

5. The polymer of claim 2, wherein the anticancer drug is selected from the group consisting of a taxane, camptotheca, and anthracycline.

6. The polymer of claim 5, wherein the camptotheca is camptothecin; wherein the anthracycline is doxorubicin; and wherein the taxane is selected from the group consisting of paclitaxel and docetaxel.

7. The polymer of claim 2, wherein the targeting agent is selected from the group consisting of an arginine-glycine-aspartate (RGD) peptide, fibronectin, folate, galactose, an apolipoprotein, insulin, transferrin, a fibroblast growth factor (FGF), an epidermal growth factor (EGF), and an antibody.

8. The polymer of claim 2, wherein the magnetic resonance imaging comprises a Gd(III) compound.

9. The polymer of claim 8, wherein the Gd(III) compound comprises:

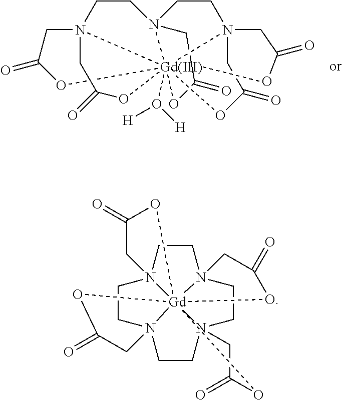

10. The polymer of claim 1, wherein R² is: 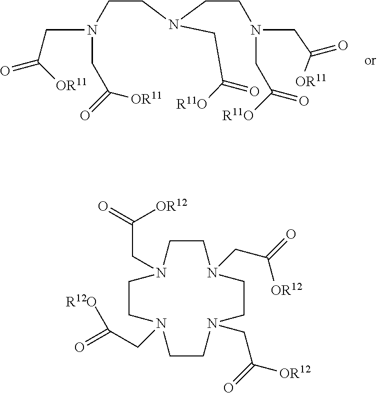
wherein each R¹¹ is independently hydrogen, ammonium, or an alkali metal;
and wherein each R¹² is independently hydrogen, ammonium, or an alkali metal.

11. The polymer of claim 1, wherein at least of one R³ and R⁴ is 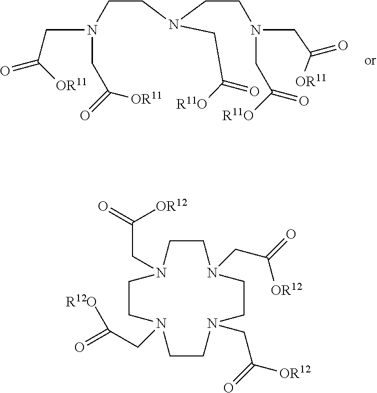
wherein each R¹¹ is independently hydrogen, ammonium, or an alkali metal;
and wherein each R¹² is independently hydrogen, ammonium, or an alkali metal.

12. The polymer of claim 1, wherein R² is:

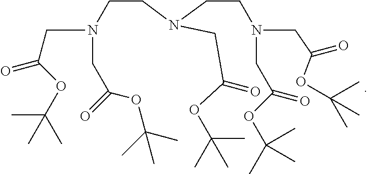

13. The polymer of claim 1, wherein at least of one R³ and R⁴ is

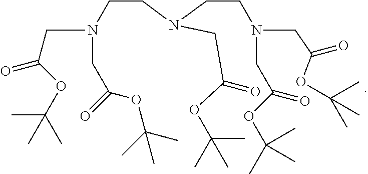

14. The polymer of claim 1, wherein the agent of the second compound is selected from the group consisting of a stabilizing agent, an optical imaging agent, an anticancer drug, a targeting agent, and a magnetic resonance imaging agent.

15. The polymer of claim 14, wherein the stabilizing agent is PEG.

16. The polymer of claim 14, wherein the optical imaging agent of the second compound is selected from the group consisting of an acridine dye, a coumarine dye, a rhodamine dye, a xanthene dye, cyanine dye, and a pyrene dye.

17. The polymer of claim 14, wherein the anticancer drug is selected from the group consisting of a taxane, camptotheca, and anthracycline.

18. The polymer of claim 17, wherein the camptotheca is camptothecin; wherein the anthracycline is doxorubicin; and wherein the taxane is selected from the group consisting of paclitaxel and docetaxel.

19. The polymer of claim 14, wherein the targeting agent is selected from the group consisting of an arginine-glycine-aspartate (RGD) peptide, fibronectin, folate, galactose, an apolipoprotein, insulin, transferrin, a fibroblast growth factor (FGF), an epidermal growth factor (EGF), and an antibody.

20. The polymer of claim 14, wherein the magnetic resonance imaging comprises a Gd(III) compound.

21. The polymer of claim 20, wherein the Gd(III) compound comprises:

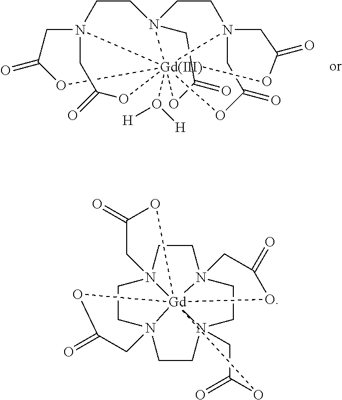

22. The polymer of claim 1, wherein R⁶ is: 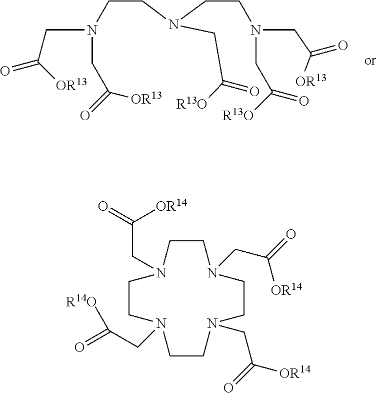
wherein each R¹³ is independently hydrogen, ammonium, or an alkali metal;
and wherein each R¹⁴ is independently hydrogen, ammonium, or an alkali metal.

23. The polymer of claim 1, wherein at least of one R⁷ and R⁸ is 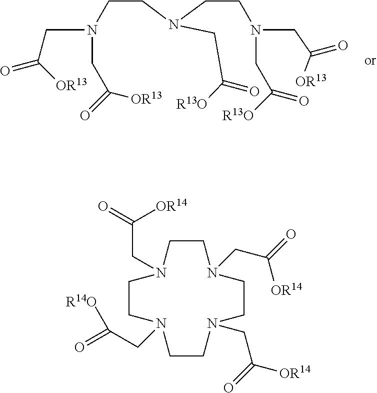
wherein each R¹³ is independently hydrogen, ammonium, or an alkali metal;
and wherein each R¹⁴ is independently hydrogen, ammonium, or an alkali metal.

24. The polymer of claim 1, wherein R⁶ is:

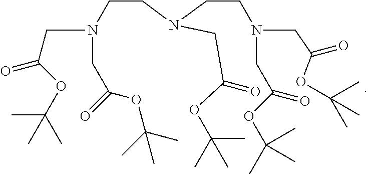

25. The polymer of claim 1, wherein at least of one R⁷ and R⁸ is

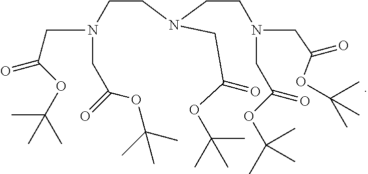

26. The polymer of claim 1, wherein the polymer comprises a total amount of agent in the range of about 1% to about 50% (weight/weight) based on the mass ratio of the total amount of agent to the polymer.

27. The polymer of claim 1, wherein R¹ and R⁵ are independently an optionally substituted C₁₋₁₀ alkyl or an optionally substituted C₆₋₂₀ aryl.

28. A pharmaceutical composition comprising the polymer of claim 1, and at least one selected from a pharmaceutically acceptable excipient, a carrier, and a diluent.

29. A method of making the polymer of claim 1, comprising:
dissolving or partially dissolving a polymeric reactant comprising at least one recurring unit selected from the group consisting of Formula (V) and Formula (VI) in a solvent to form a dissolved or partially dissolved polymeric reactant; 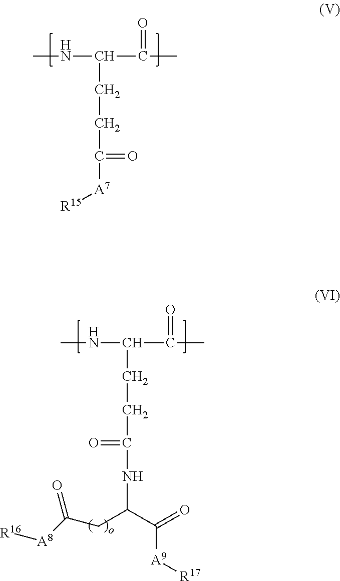
wherein:
o is independently 1 or 2;
A⁷, A⁸, and A⁹ are oxygen; and
R¹⁵, R¹⁶ and R¹⁷ are independently selected from the group consisting of hydrogen, ammonium, and an alkali metal; and
reacting the dissolved or partially dissolved polymeric reactant with a second reactant, wherein the second reactant comprises a compound of Formula (VII): 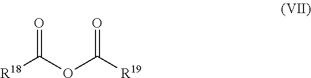
wherein R¹⁸ and R¹⁹ are independently selected from the group consisting of an optionally substituted C₁₋₁₀ alkyl and an optionally substituted C₆₋₂₀ aryl.

30. A method of treating, ameliorating, or diagnosing a disease or condition comprising administering an effective amount of the polymer of claim 1.

31. A method of imaging a portion of tissue comprising contacting a portion of tissue with an effective amount of the polymer of claim 1.
